(19) United States
(12) Patent Application Publication
(10) Pub. No.: US 2011/0270256 A1
(43) Pub. Date: Nov. 3, 2011

(54) SURGICAL RASP WITH RADIOFREQUENCY ABLATION

(75) Inventors: Keith J. Nelson, Logan, UT (US); M. Mary Sinnott, Logan, UT (US); Nathan D. Hansen, Hyde Park, UT (US); Joseph Q. Marietta, Hyde Park, UT (US); Douglas M. Lorang, North Logan, UT (US); Daniel F. Justin, Orlando, FL (US); Trevor Lewis, Lehi, UT (US)

(73) Assignees: MEDICINELODGE, INC. DBA IMDS CO-INNOVATION, Logan, UT (US); Keith J. Nelson, Logan, UT (US)

(21) Appl. No.: 13/102,351

(22) Filed: May 6, 2011

Related U.S. Application Data
(63) Continuation-in-part of application No. 12/765,451, filed on Apr. 22, 2010.
(60) Provisional application No. 61/245,487, filed on Sep. 24, 2009.
(60) Provisional application No. 61/332,308, filed on May 7, 2010.
(60) Provisional application No. 61/382,795, filed on Sep. 14, 2010.

(51) Int. Cl. A61B 17/16

(52) U.S. Cl. 606/85

(57) ABSTRACT

A surgical rasping system functional in multiple orthopedic applications, including but not limited to shoulder, knee, hip, wrist, ankle, spinal, or other joint procedures. The system comprises a rasping head which may be low profile and offer a flat cutting/rasping surface, and is configured to be driven by an attached hub that translates a rotational movement into a reciprocating motion. Suction for removal of bone fragments or other tissues is provided through an opening spaced apart from or adjacent to the rasping surface. A radiofrequency ablation (RF) electrode may be carried on the rasping system to provide ablation or coagulation of soft tissues.

### CROSS-REFERENCE TO RELATED APPLICATIONS

This application is a continuation-in-part of:

pending U.S. application Ser. No. 12/765,451, filed Apr. 22, 2010, which carries Applicant's docket No. NEL-1, and is entitled SURGICAL RASPING SYSTEMS AND METHODS, which is a non-provisional of:

U.S. Provisional Patent Application No. 61/245,487, filed Sep. 24, 2009, which carries Applicants' docket no. NEL-1 PROV, and is entitled SURGICAL RASPING SYSTEM.

This application is also a non-provisional of:

pending U.S. Provisional Patent Application No. 61/332,308, filed May 7, 2010, which carries Applicants' docket no. NEL-2 PROV, and is entitled RECIPROCATING RASP WITH RF ABLATION PROBE; and

pending U.S. Provisional Patent Application No. 61/382,795, filed Sep. 14, 2010, which carries Applicants' docket no. NEL-8 PROV, and is entitled RECIPROCATING SURGICAL INSTRUMENTS WITH ADDED FUNCTIONALITY.

The above-identified documents are incorporated herein by reference.

### FIELD OF THE INVENTION

This invention relates to surgical tissue removal devices by which anatomical tissues may be cut and removed from a joint or other operative site. Specifically, this invention relates to rasping instruments having reciprocating motion and suction.

### BACKGROUND OF THE INVENTION

Surgical procedures including subacromial decompression, arthroscopic resection of the acromioclavicular joint (also known as the Mumford procedure), and anterior cruciate ligament reconstruction involving notch plasty, may all necessitate removal of osteophytes. Other conditions such as chondromalacia and osteochondritis dissecans may call for removal of osteophytes or chondrocytes. It is known to use shavers and burrs having rotational cutting surfaces to remove these hard tissues. However, the round cutting surface of a shaver or bun system is not advantageous to creating or preparing a flat surface. The forces applied while using a rotational round cutting surface tend to pull the cutting end to either side by a moment force pivoting on the hand making precise control difficult. Working in confined spaces may exacerbate these issues, as adjacent soft tissues may easily be grabbed by a rotating cutting surface. Therefore, the need exists for an instrument with a reciprocating, flat cutting surface to provide a surgeon with greater control over the instrument and enhanced ability to create/prepare a flat tissue surface, especially in confined areas.

Removal and/or coagulation of soft tissues adjacent to articular joints is often necessary to gain access to the joint space. For example, in a hip or shoulder arthroscopy procedure, the ligaments forming the joint capsule may need to be resected or penetrated to clear a pathway for a surgical instrument to reach the joint. Radiofrequency (RF) ablation may be used to ablate these soft tissues. However, use of an independent RF probe may require an additional surgical portal for insertion of the probe, thus potentially increasing tissue trauma, pain, and healing time. In other procedures such as non-minimally invasive orthopedic procedures, an independent RF probe represents an additional instrument, thus potentially increasing procedure time, cost, and complexity.

### BRIEF DESCRIPTION OF THE DRAWINGS

Various embodiments of the present invention will now be discussed with reference to the appended drawings. It is appreciated that these drawings depict only typical embodiments of the invention and are therefore not to be considered limiting of its scope.

FIG. 1A is an isometric view of a reciprocating rasping system;

FIG. 1B is a front view of the rasping system of FIG. 1A in a retracted configuration;

FIG. 1C is a front view of the rasping system of FIG. 1A in an extended configuration;

FIG. 2 is an isometric view of the rasping system of FIG. 1A with an outer housing and collet removed, and a longitudinal axis of the rasping system;

FIG. 3A in an enlarged isometric view of a rasp head of the rasping system of FIG. 1A; FIG. 3B is a front view of the rasp head of FIG. 3A; FIG. 3C is a side view of the rasp head of FIG. 3C; FIG. 3D is a cross-sectional view of the rasp head of FIG. 3A taken along section line D-D;

FIG. 4A is a iso-side view of a tissue removal member of the rasping system of FIG. 1A; FIG. 4B is an enlarged side view of a shaft key of the tissue removal member of FIG. 4A;

FIG. 5A is a side view of an outer housing of the rasping system of FIG. 1A; FIG. 5B is cross-sectional view of the outer housing of FIG. 5A, taken along line B-B of FIG. 5C; FIG. 5C is a top end view of the outer housing of FIG. 5A; FIG. 5C is a bottom end view of the outer housing of FIG. 5A;

FIG. 6A is an isometric view of a first side of a driving hub of the rasping system of FIG. 1A;

FIG. 6B is an isometric view of a second side of a driving hub of the rasping system of FIG. 1A;

FIG. 7A is an isometric view of a spring collet and spring of the rasping system of FIG. 1A; FIG. 7B is cross-sectional view of the spring collet and spring along section line B-B of FIG. 7C; FIG. 7C is a bottom end view of the spring collet and spring of FIG. 7A;

FIG. 8 is a longitudinal cross-sectional view of a handle portion and a segment of a shaft portion of the rasping system of FIG. 1A in the retracted position;

FIG. 9 is a longitudinal cross-sectional view of a handle portion and a segment of a shaft portion of the rasping system of FIG. 1A in the extended position;

FIG. 10 is an isometric view of the rasping system of FIG. 1A coupled in an exemplary powered handpiece;

FIG. 11A is an isometric view of an alternate embodiment of a rasp head, a tissue removal portion angled relative to the remainder of the rasp head; FIG. 11B is a side view of the rasp head of FIG. 11A;

FIG. 12A is an isometric view of an alternate embodiment of a rasp head comprising a convex tissue removal surface; FIG. 12B is a cross-sectional view of the rasp head of FIG. 12A taken along line B-B;

FIG. 13A is an isometric view of an alternate embodiment of a rasp head comprising elongated rasping teeth; FIG. 13B is a side view of the rasp head of FIG. 13A;

FIG. 14A is an isometric view of an alternate embodiment of a rasp head comprising a reduced tissue removal surface; FIG. 14B is a side view of the rasp head of FIG. 14A;

FIG. 15A is an isometric view of an alternate embodiment of a rasp head comprising a crescent-shaped tissue removal surface; FIG. 15B is a side view of the rasp head of FIG. 15A;

FIG. 16A is an isometric view of an alternate embodiment of a rasp head comprising a concave removal surface; FIG. 16B is a side view of the rasp head of FIG. 16A;

FIG. 17A is an isometric view of an alternate embodiment of a rasp head comprising bi-directional rasping teeth; FIG. 17B is a side view of the rasp head of FIG. 17A;

FIG. 18A is an isometric view of an alternate embodiment of a rasp head comprising a suction pathway opening on a back side of the head; FIG. 18B is a side view of the rasp head of FIG. 18A;

FIG. 19A is a side view of head and shaft portions of an RF/reciprocating rasp device including a rasp tissue removal surface, an ablation electrode integral with the tissue removal member, an insulating layer, and a return electrode; FIG. 19B is a top view of the device of FIG. 19A; FIG. 19C is a cross-sectional side view of the device of FIG. 19A;

FIG. 20 is an exploded isometric view of the device of FIG. 19A including a tissue removal member which is integral with the ablation electrode, the insulating layer, the return electrode, and an outer sleeve;

FIG. 21A is a side view of head and shaft portions of an RF/reciprocating rasp device including a tissue removal member comprising a rasp head having a tissue removal surface, an ablation electrode positioned on a back side of the rasp head, an insulating layer, and a return electrode integral with the tissue removal surface, and an outer sleeve; FIG. 21B is a top view of the device of FIG. 21A; FIG. 21C is a cross-sectional side view of the device of FIG. 21A;

FIG. 22 is an exploded side view of the device of FIG. 21A including the rasp head and inner shaft integral with the return electrode, the insulating layer, the ablation electrode, and the outer sleeve;

FIG. 23A is a side view of head and shaft portions of an RF/reciprocating rasp device including a tissue removal member comprising a rasp head having a tissue removal surface, an outer sleeve having an extension, an ablation electrode positioned on a first side of the sleeve extension, a return electrode integral positioned on a second side of the sleeve extension, and an insulating layer; FIG. 23B is a top view of the device of FIG. 23A; FIG. 23C is a cross-sectional side view of the device of FIG. 23A;

FIG. 24 is an exploded side view of the device of FIG. 23A;

FIG. 25A is a bottom view of a reciprocating rasp device including an auxiliary device; FIG. 25B is an enlarged view of a head portion of the device of FIG. 25A; FIG. 25C is a cross-sectional view of a shaft portion of the device of FIG. 25A taken along line C-C;

FIG. 26A is a top isometric view of a reciprocating rasp device including an auxiliary device, RF ablation system, and an infusion system; FIG. 26B is a top view of the device of FIG. 26A, FIG. 26C is a side isometric view of the device of FIG. 26A;

FIG. 27 is an isometric view of an alternate embodiment of a reciprocating rasping rasping system including a head portion, a shaft portion, and a handle portion;

FIG. 28A is a side view of the rasping system of FIG. 27, with an outer housing removed; FIG. 28B is a bottom view of the rasping system of FIG. 24A;

FIG. 29 is an exploded view of the rasping system of FIG. 27;

FIG. 30 is a cross-sectional view of the handle and shaft portions of the reciprocating rasping system of FIG. 27 with a tissue removal member in an retracted position;

FIG. 31 is a cross-sectional view of the handle and shaft portions of the reciprocating rasping system of FIG. 27 with a tissue removal member in an extended position;

FIG. 32A is an isometric view of a rotatable cam of the system of FIG. 27; FIG. 32B is a cross-sectional view of the rotatable cam of FIG. 32A taken along line B-B; FIG. 32C is an end view of the rotatable cam of FIG. 32A;

FIG. 33A is an isometric view of a fixed cam of the system of FIG. 27; FIG. 33B is a cross-sectional view of the fixed cam of FIG. 33A taken along line B-B; FIG. 33C is an end view of the fixed cam of FIG. 33A.

### DETAILED DESCRIPTION OF THE PREFERRED EMBODIMENTS

The present invention relates to tissue removal devices and methods by which body tissues may be cut and removed during surgery. Those of skill in the art will recognize that the following description is merely illustrative of the principles of the invention, which may be applied in various ways to provide many different alternative embodiments. This description is made for the purpose of illustrating the general principles of this invention and is not meant to limit the inventive concepts in the appended claims.

The present invention provides a rasping system that is shaped such that it is functional in multiple orthopedic surgery applications, including but not limited to shoulder, knee, hip, wrist, ankle, spinal, or other joint procedures. The system comprises a rasping head which may be low profile and offer a flat cutting/rasping surface, and is configured to be driven by an attached hub that will translate a rotational movement into a reciprocating motion. Suction for removal of bone fragments or other tissues may be provided through an opening in or adjacent the rasping head.

This device provides an alternative method of removing hard tissue to the currently used shavers and burrs that offer a rotational cutting surface. By applying a reciprocating flat cutting surface the surgeon has greater control over the instrument and is better able to create/prepare a flat surface. The reciprocating force of the rasp applies resisting pressure to the surgeons hand in the axial direction with the hand, making control much easier. Increased control will result in a decrease in injury to the surrounding soft tissue. The rasp also has a lower profile than many of the existing shaver systems allowing access to tight joints without damaging surrounding tissues. The teeth of the rasp may be positioned such that the cut material will be pulled towards the suction pathway to more efficiently remove debris from the surgical site, thus decreasing the duration of a procedure.

According to a first aspect, the present invention provides a tissue removal device for being driven by a powered rotary handpiece, including: a rotary hub; a tissue removal member comprising a head portion having a first side and a second side opposite the first side, a tissue removal surface located on the first side, the tissue removal member coupled in sliding contact with the rotary hub; a motion conversion mechanism, wherein when the rotary hub is rotated about an axis, the motion conversion mechanism urges motion of the tissue removal member along the axis, wherein the motion consists of reciprocating translation between a first retracted position and a second extended position; and a radiofrequency ablation system carried on the tissue removal device for ablation or coagulation of soft tissues, the radiofrequency ablation system including an ablating electrode and a return electrode.

In an embodiment, the radiofrequency ablation system further includes an insulation layer positioned between the ablating electrode and the return electrode.

In an embodiment, the radiofrequency ablation system is carried on the tissue removal member. In an embodiment, the head portion first side carries the ablating electrode. In an embodiment, the tissue removal surface is the ablating electrode.

In an embodiment, the head portion second side carries the ablating electrode. In an embodiment, the tissue removal surface is the return electrode.

In an embodiment, the tissue removal device further includes a suction pathway extending from a distal opening on the head portion to a proximal opening, the distal opening located on the same head side as the ablating electrode. In an embodiment, the distal opening is located on the opposite head side from the ablating electrode.

In an embodiment, the tissue removal device further includes a sleeve member having a distal end and a proximal end, the tissue removal member extending through the sleeve member, wherein the radiofrequency ablation system is carried on the sleeve member. In an embodiment, the tissue removal device further includes a suction pathway extending from a distal opening on the head to a proximal opening.

In an embodiment, the ablating electrode further includes an active ablation portion and the return electrode further includes an active return portion, wherein the active return portion is at least three times greater than the active ablation portion.

In an embodiment, the radiofrequency ablation system further includes a controller which selectively controls transmission of radiofrequency current through the radiofrequency ablation system, wherein the transmission of radiofrequency current through the radiofrequency ablation system for ablation or coagulation of soft tissues is independent of the reciprocating translation motion of the tissue removal member.

In an embodiment, the tissue removal surface comprises a plurality of teeth for removing hard tissue.

According to a second aspect, the present invention provides a tissue removal device for being driven by a powered rotary handpiece, including: a rotary hub; a tissue removal member comprising tissue removal means, the tissue removal member coupled in sliding contact with the rotary hub; means for motion conversion, wherein when the rotary hub is rotated about an axis, the means for motion conversion urges motion of the tissue removal member along the axis, wherein the motion consists of reciprocating translation between a first retracted position and a second extended position; and means for soft tissue ablation or coagulation carried on the tissue removal device.

In an embodiment, the tissue removal device further includes means for suction carried on the tissue removal device.

In an embodiment, the means for soft tissue ablation or coagulation is carried on the tissue removal member.

In an embodiment, the tissue removal device further includes a sleeve member having a distal end and a proximal end, the tissue removal member extending through the sleeve member, wherein the means for soft tissue ablation or coagulation is carried on the sleeve member.

According to a third aspect, the present invention provides a method for removal of hard and soft body tissues at a surgical site using a single instrument, the method including: providing a tissue removal device for being driven by a powered rotary handpiece, the tissue removal device comprising: a rotary hub, a tissue removal member comprising a tissue removal surface, the tissue removal member coupled in sliding contact with the rotary hub, a motion conversion mechanism, wherein when the rotary hub is rotated about an axis, the motion conversion mechanism urges motion of the tissue removal member along the axis, wherein the motion consists of reciprocating translation between a first retracted position and a second extended position, and a radiofrequency ablation system comprising an ablating electrode and a return electrode; positioning the tissue removal surface adjacent hard tissue to be removed; powering the tissue removal device to provide the reciprocating translation of the tissue removal surface to remove a selected amount of hard tissue; positioning the ablating electrode adjacent soft tissue to be ablated or coagulated; and powering the radiofrequency ablation system to ablate or coagulate a selected amount of soft tissue by radiofrequency ablation.

In an embodiment, the tissue removal device further comprises a suction pathway, and the method further includes applying suction to suction the removed tissue through the suction pathway away from the surgical site.

In an embodiment, the method further includes powering the radiofrequency ablation system to ablate or coagulate soft tissue while simultaneously powering the tissue removal device to provide reciprocating translation of the tissue removal surface.

In an embodiment, the ablating electrode is carried on the tissue removal member, the tissue removal member including a head portion having first side and a second side opposite the first side, the tissue removal surface located on the first side. In an embodiment, the ablating electrode is carried on the tissue removal surface and the method further includes positioning the tissue removal surface adjacent soft tissue to be ablated or coagulated. In an embodiment, the ablating electrode is carried on the head portion second side and the method further includes positioning the head portion second side adjacent soft tissue to be ablated or coagulated.

In an embodiment, wherein the tissue removal device further comprises a sleeve member having a distal end and a proximal end, the tissue removal member extending through the sleeve member, the radiofrequency ablation system carried on the sleeve member, and the method further includes positioning the sleeve member adjacent soft tissue to be ablated or coagulated.

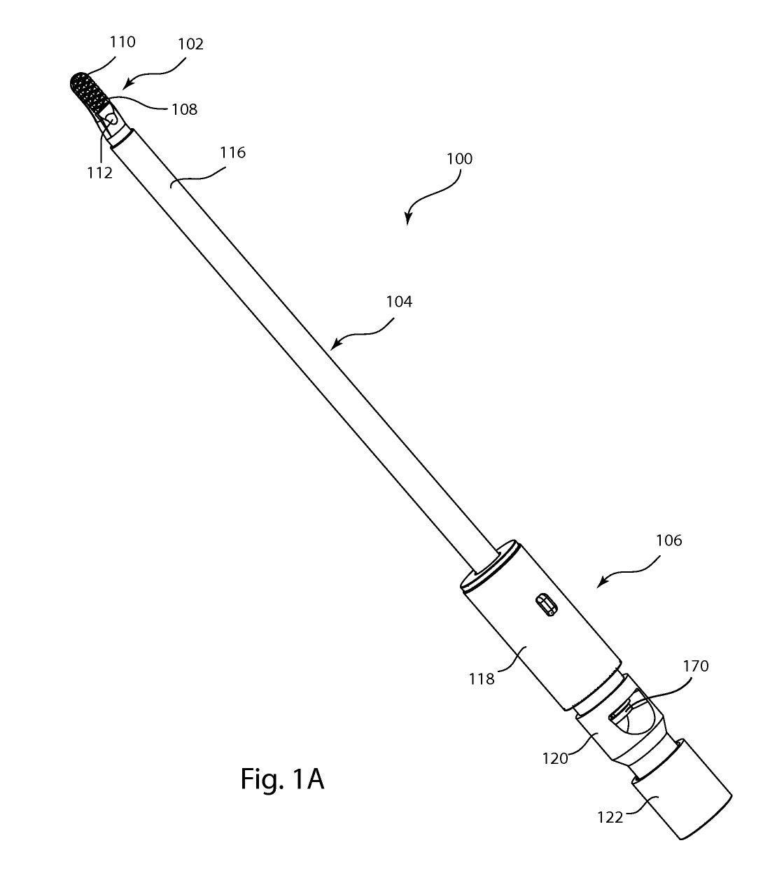

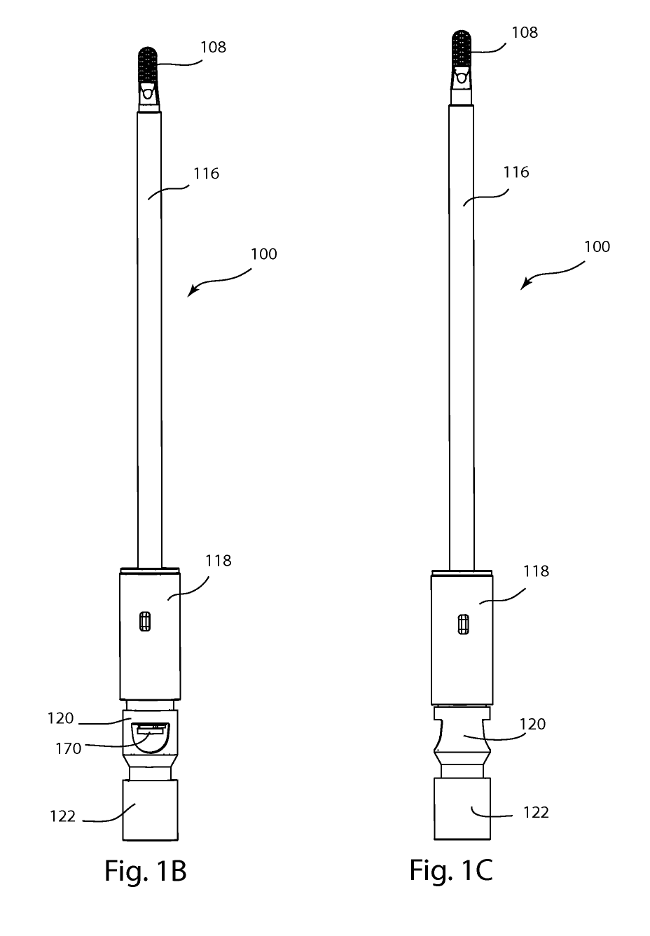

Referring to FIG. 1A, rasp system 100 is shown in an isometric view. Rasp system 100 comprises head portion 102, shaft portion 104, and handle portion 106. Head portion 102 comprises rasp head 108, which includes a plurality of teeth 110 or cutting edges which may cut anatomical tissues when drawn along the tissue surface. The teeth may be particularly suited for cutting or removing hard tissues such as bone or cartilage. A suction opening 112 is located on the head portion 102, and may be disposed between the teeth and the shaft portion. The shaft portion 104 comprises inner shaft 114 (not visible in FIG. 1A) which extends proximally from the rasp head 108 and is received in the handle portion 106. The inner shaft 114 extends through an optional outer sleeve 116 which is joined to the handle portion 106. At its proximal end, inner shaft 114 is received within a shaft key 170 (not visible in FIG. 1A).

Handle portion 106 includes an outer housing 118, a driving hub 120, and a spring collet 122 which houses a spring 250 (not visible in FIG. 1). Outer housing 118 comprises a cam surface (not visible in FIG. 1) which is complementarily shaped to a cam follower surface on driving hub 120. When handle portion 106 is engaged in a powered rotary handpiece and power is supplied, hub 120 rotates, and the cam and cam follower surfaces provide a motion conversion mechanism which converts the rotary motion of the hub to axial reciprocal motion of the inner shaft 114 and attached head 108. Rasp system 100 is connectable via spring collet 122 to a powered handpiece, to provide rotary power to the rasp system, and to provide suction. Suitable handpieces include the Linvatec Advantage Shaver (Ref D9824) or another similar system known in the art.

FIG. 1B illustrates rasp system 100 in a retracted configuration. In this configuration, the shaft key 170, inner shaft 114 (not visible; within outer sleeve 116) and rasp head 108 have been pulled by interaction of the cam and cam follower surfaces to a proximal position. FIG. 1C illustrates rasp system 100 in an extended configuration. In this configuration, driving hub 120 has rotated relative to the outer housing 120; and the shaft key, inner shaft 114 and rasp head 108 have been reciprocally translated to a distal position by the spring bias of spring 250. It is appreciated that an alternate embodiment of the invention may include a curved inner shaft and, optionally, a curved outer sleeve. In the curved embodiment the rasp head may be angled relative to the inner shaft, and the outer sleeve may be sized to allow free reciprocation of the inner shaft.

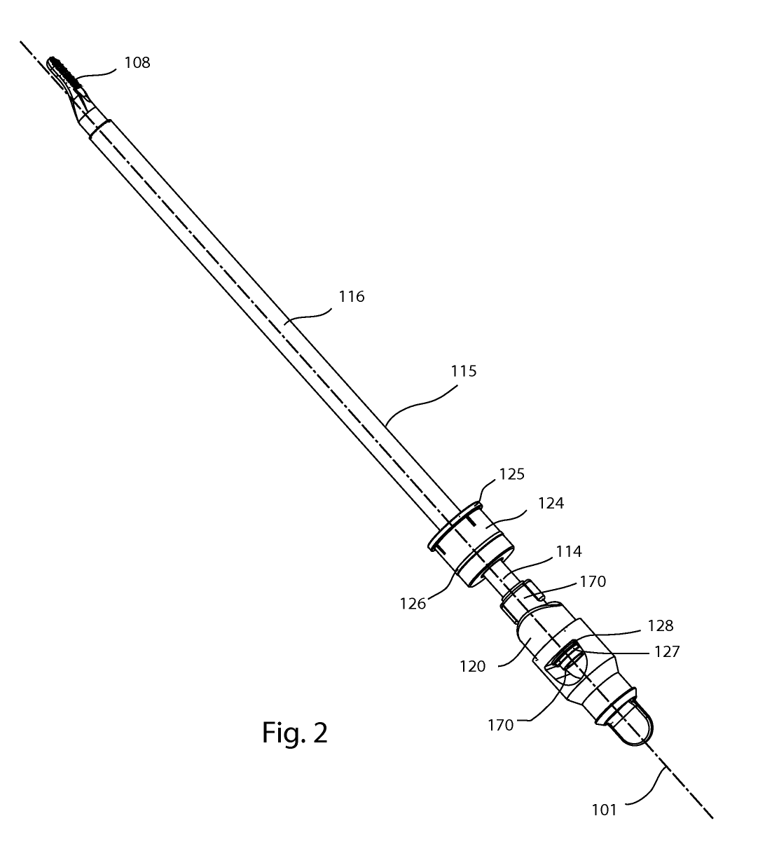

FIG. 2 illustrates rasp system 100 minus the outer housing 118 and spring collet 122. Outer sleeve 116 is joined to plug 124. Plug 124 comprises a rim 125 and a protruding ring 126. When received within the outer housing 118 as in FIG. 1, ring 126 may provide a snap connection with a groove feature within outer housing 118, and rim 125 may seat against a distal end of the outer housing. Once joined with the outer housing 118, plug 124 and outer sleeve do not translate or rotate relative to the outer housing. The outer sleeve 116 provides protection to surrounding tissues when rasp system 100 is used; outer sleeve 116 does not rotate or reciprocate, yet allows reciprocal movement of inner shaft 114 within. Space between the inner shaft 114 and the outer sleeve 116 may optionally be lubricated. Together, the rasp head 108, inner shaft 114 and shaft key 170 comprise a tissue removal member 115.

Proximal to the plug 124, the inner shaft 114 is received in the shaft key 170 and is non-movable relative to the shaft key. A portion of shaft key 170 is received within a portion of hub 120, which is rotatable about the shaft key. A snap ring 127 is received in a groove formed at the proximal end of the shaft key, and retains the shaft key 170 within the hub 120 while still allowing the hub 120 to rotate about the shaft key. A washer 128 is positioned around the shaft key 170 between the snap ring 127 and the hub 120. The system 100 comprises a longitudinal axis 101 about which the hub 120 rotates, and along which the tissue removal member 115 is reciprocally translated.

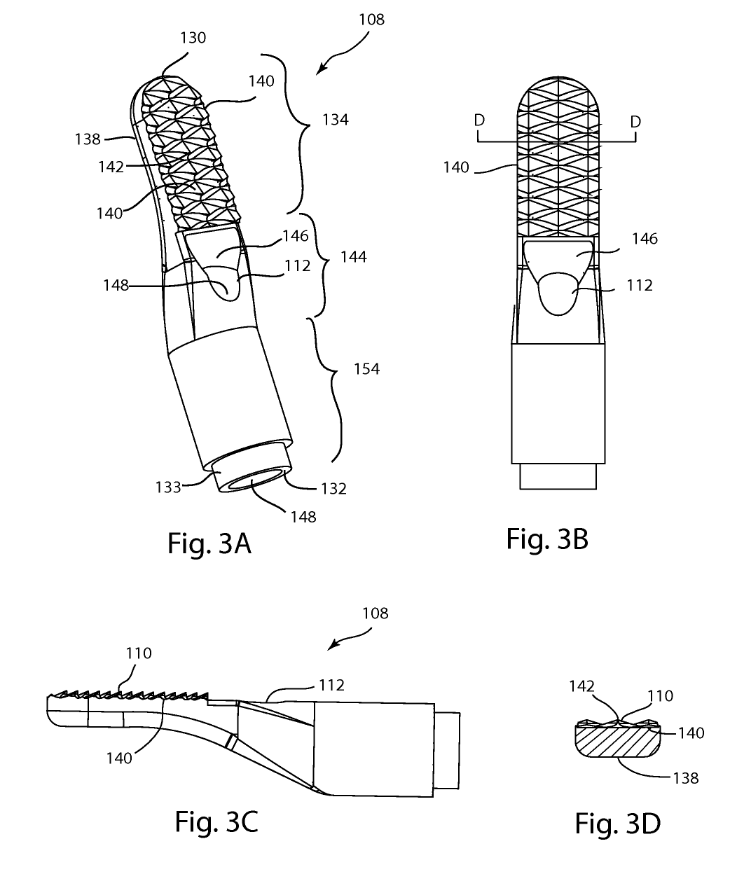

Referring to FIGS. 3A through 3D, several views of rasp head 108 are shown. Rasp head 108 comprises a distal end 130, and a proximal end 132, and further comprises a working portion 134, a head transition portion 144 and a head shaft portion 154. The working portion 134 comprises a first side 136 which may be also be known as a front side, and a second, or back side 138 opposite the first side. A tissue removal surface 140 is disposed on the first side 136, although it is appreciated that in alternate embodiments, the tissue removal surface may be disposed on the back side, or on both the front and back sides. The tissue removal surface 140, may be flat as in FIGS. 3A-3D, or in other embodiments may be concave or convex. The plurality of teeth 110 populates the tissue removal surface, each tooth having a cutting portion 142. The cutting portion 142 may be a point as seen in the teeth depicted in FIGS. 2A-2D, but in other embodiments the cutting portion may be an edge, or a combination of one or more edges and a point. The teeth may be distributed individually; in even ranks or rows; or in alternate ranks or rows. In alternative embodiments of the cutting head, the number, size, and distribution of the teeth may vary to provide a variety of tissue cutting surfaces suitable for different tissue removal procedures. The cutting portions 142 may be uni-directionally oriented as in FIGS. 3A-3D, meaning that all of the teeth point the same direction. Advantageously, the teeth may be pointed toward the suction opening 112, thus facilitating efficient movement of cut debris into the suction opening. Another feature of uni-directional teeth is that the teeth may only cut into tissue when the rasp head is moved in one direction; for example if the teeth are pointed proximally, cutting will occur when the rasp head is translated proximally.

The transition portion 144 extends between the working portion and the head shaft portion, and may be angled relative to the working and/or head shaft portions. Proximal to and spaced apart from the tissue removal surface, the suction opening 112 provides a distal opening to a suction pathway. A fan-like scoop portion 146 adjacent the suction opening 112 may funnel excised tissue toward the suction opening. A head suction bore 148 extends proximally from the suction opening 112, forming a portion of the suction pathway.

The head shaft portion 154 extends from the transition portion 144 to the proximal end 132 of the rasp head 108. At the proximal end 132, a fitting or connection feature 133 allows for joining of the rasp head 108 to the inner shaft 114. The head suction bore 148 terminates at the proximal end 132, but the suction pathway continues through the hollow inner shaft 114. The rasp head 108 may be removably joined to the inner shaft via a press fit or mechanical fit, or may be permanently joined via a weld or other permanent connection.

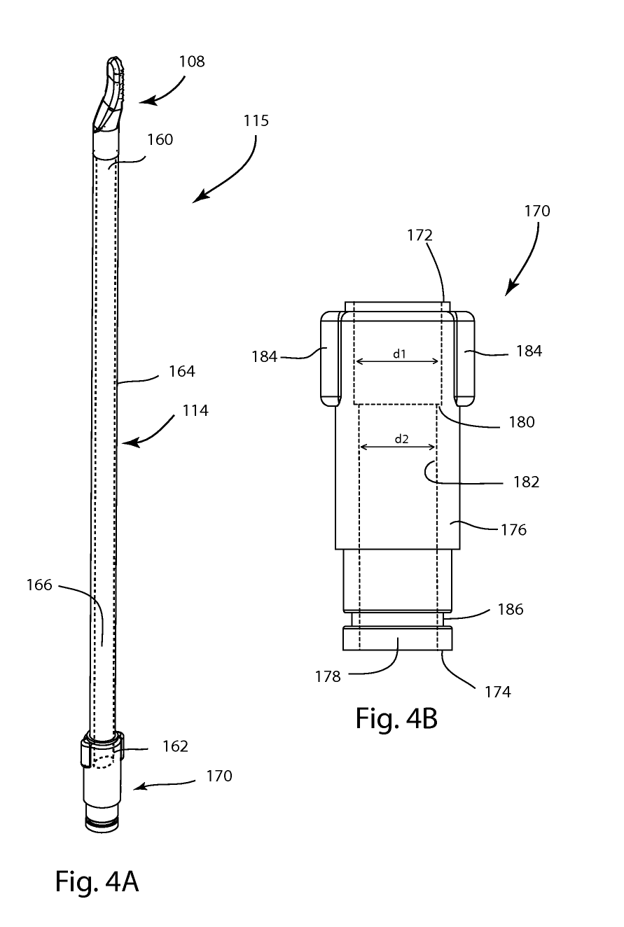

FIG. 4A illustrates the rasp head 108, inner shaft 114, and a shaft key 170, which together comprise the tissue removal member 115. The inner shaft 114 comprises a tubular member having a distal end 160, a proximal end 162 and an inner shaft body 164 extending therebetween. The inner shaft body defines an inner shaft bore 166, indicated by dashed lines, extending from the distal end to the proximal end, forming a portion of the suction pathway. The proximal end 162 of the inner shaft is received in the shaft key 170. Inner shaft 114 may be glued, welded, bonded, press fit or otherwise permanently joined to shaft key 170, so that no movement including translation or rotation between inner shaft 114 and shaft key 170 is allowed. Inner shaft 114 may be monolithically formed with shaft key 170.

Referring to FIG. 4B, shaft key 170 comprises a distal end 172, a proximal end 174, and generally cylindrical key body 176 extending therebetween. A key bore 178 (indicated by dashed lines) extends the length of the shaft key, and forms a portion of the suction pathway. At its distal end, the key bore has a first diameter d1 dimensioned to receive the proximal end of the inner shaft 114. Proximal to a shoulder 180 formed in an inner wall 182 of the key body 176, the key bore has a second diameter d2. Two individual wings 184 protrude from the key body 176, opposite from one another near the distal end 172. The wings 184 are shaped to be received in recesses formed within the outer housing, preventing rotation of the tissue removal member when the hub is rotated. It is appreciated that in other embodiments of the invention, the number and placement of the wings 184 may vary, or the wings may be formed on the outer housing, to be received in recesses formed on the shaft key 170. Toward the proximal end 174 of the shaft key 170, an annular groove 186 is formed on the outside of the key body 176. The groove 186 is shaped to receive snap ring 127. The suction pathway comprises the continuous pathway formed by head suction bore 148, inner shaft bore 166 and key bore 178.

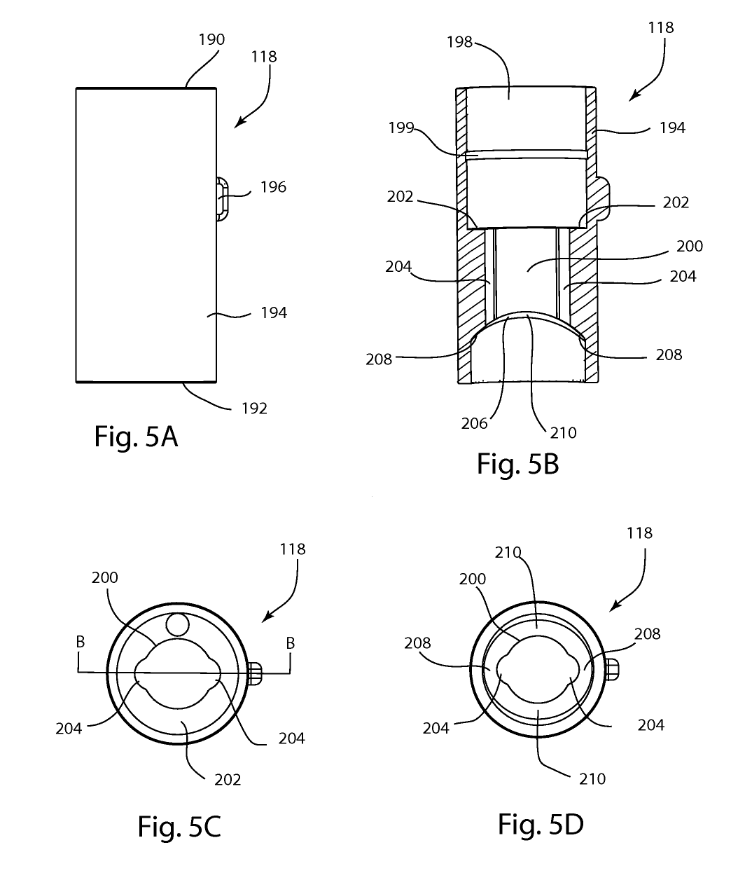

Outer housing 118 is illustrated in FIGS. 5A through 5D. The tissue removal member 115 is receivable in the outer housing, while the outer housing is shaped to be received in a powered handpiece. Outer housing 118 is generally cylindrical and comprises a distal end 190, a proximal end 192 and an outer housing body 194 extending therebetween. A tab 196 protrudes exteriorly from the outer housing body, and is shaped to be received in a groove formed in a powered handpiece, to both properly align the rasp system 100 within the handpiece and prohibit rotation of the outer housing 118 relative to the handpiece.

FIG. 5B is a longitudinal cross-sectional view of the housing, taken along line B in FIG. 5C. Extending longitudinally through the housing is housing bore 198. Toward the distal end of the housing, bore 198 is shaped to receive the generally cylindrical plug 124 (not shown) which in turn receives the outer sleeve 116. An annular inner groove 199 is shaped to fit around the ring 126 on the outer surface of the plug. An annular shoulder 202 is formed in the inner wall of the housing body 194. A keyway, or key portion 200 of the housing bore 198 is constricted, and shaped to receive a portion of the shaft key 170. Two recesses 204 in the key portion 200 are shaped to complementarily fit the wings 184 of the shaft key 170. When the shaft key 170 is received in the key portion 200 of the housing 118, the complementary fit of the wings 184 in the recesses 204 prohibits rotation of the shaft key 170, and thus tissue removal member 115, relative to the outer housing 118, but allows proximal-distal/distal-proximal translation of the shaft key 170 relative to the outer housing.

Referring to FIG. 5D, a bottom end view shows an undulating, annular cam surface 206 formed in the inner wall of the housing body 194. The annular cam surface 206 comprises two lobes 208, formed as two portions which protrude proximally, parallel to the longitudinal axis, on opposite sides of the bore 198 from one another. At the lobes 208, cam surface 206 slopes proximally from its outer diameter to its inner diameter. The lobes 208 are evenly interspersed with two hollows 210, such that, when viewed from the side, the annular cam surface 206 undulates evenly between two low points at the lobes 208, and two high points at the hollows 210.

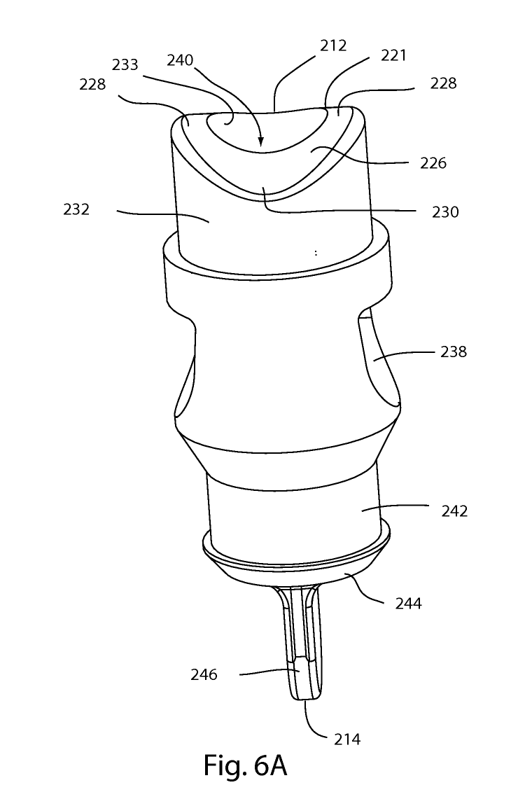

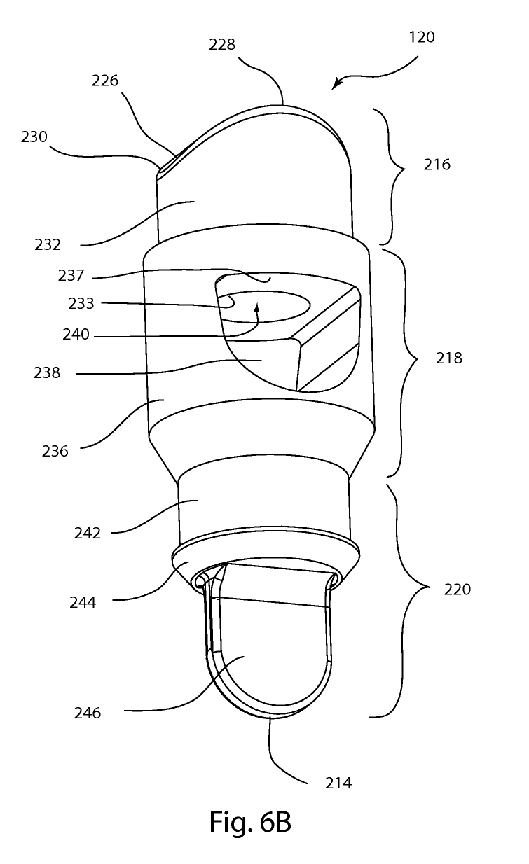

The driving hub 120 is illustrated in FIGS. 6A and 6B. The hub 120 extends longitudinally between a distal end 212 and a proximal end 214. The hub 120 comprises three portions: a distal cam portion 216, an intermediate portion 218, and a proximal driving portion 220. At the distal end 212, the hub terminates in a distal end face 221 having a cam follower surface 226 which is shaped complementarily to the cam surface 206. The cam follower surface comprises two follower lobes 228 interspersed with two follower hollows 230. At the follower hollows 230, cam follower surface 226 slopes proximally from its outer diameter to its inner diameter. The follower lobes 228 are evenly interspersed with the follower hollows 230, such that, when viewed from the side, the cam follower surface 226 undulates evenly between two low points at the hollows 230, and two high points at the lobes 228. The distal cam portion 216 is circumscribed by an annular outer wall 232. A driving hub bore 240, lined by an annular inner wall 233, extends longitudinally through the distal cam portion 216.

The intermediate portion 218 of the hub 120 comprises an intermediate body 236, through which an aperture 238 extends transversely. The driving hub bore 240 continues longitudinally from the distal cam portion 216 and terminates at a proximal hub face 237, in communication with the aperture 238. The driving hub bore 240 forms the proximal portion of the suction pathway, which terminates as it opens into the aperture.

The driving portion 220 of the driving hub 120 provides a connection feature for connection to a powered handpiece. The driving portion 220 comprises a smooth, cylindrical hub body 242 which terminates at an annular flange 244. The flange 244 forms a lip extending exteriorly from the hub body. Proximal to the hub body and flange, a plate-like driving tab 246 projects longitudinally, and transversely across the diameter of the hub body. The driving tab 246 is shaped to be coupled with a driver in the powered handpiece, to provide rotational motion to the driving hub. It is appreciated that in other embodiments of the invention, the connection to the powered handpiece may take other forms, including but not limited to a square, star, cross, X-shape, H-shape, or other form compatible with the handpiece.

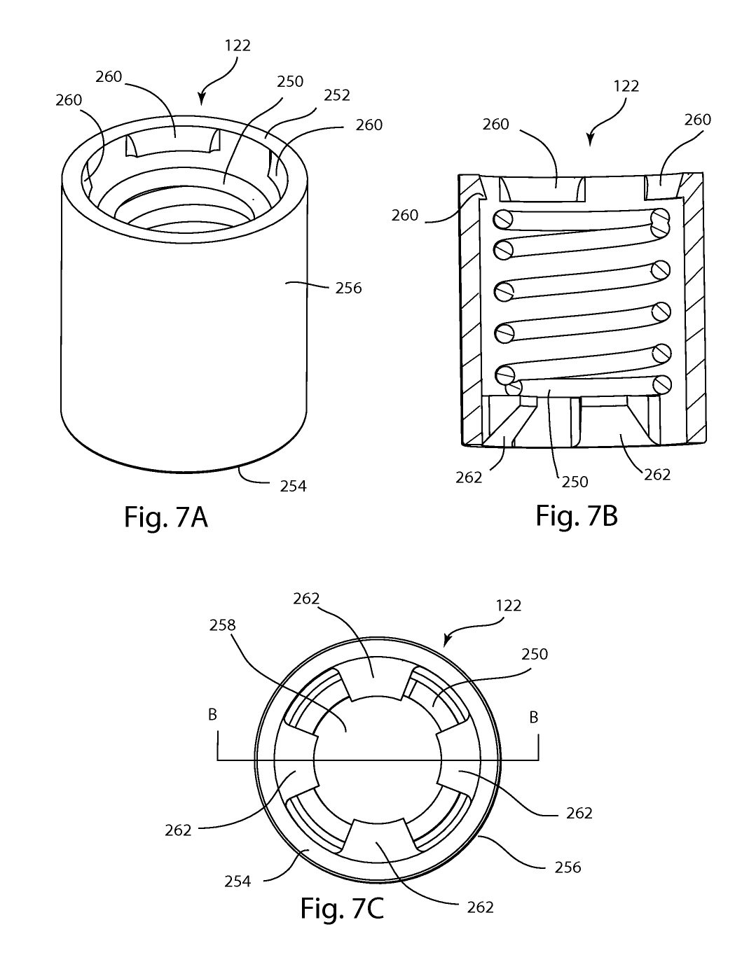

Referring to FIGS. 7A through 7C, the spring collet 122 and a spring 250 are illustrated. Spring collet 122 is generally cylindrical and tubular in form, comprising a distal end 252, a proximal end 254, and a tubular collet body 256 extending therebetween. A collet bore 258 is defined and surrounded by the collet body 256. Adjacent the distal end 252, a plurality of distal stops 260 formed on the collet body 256 protrude inward into the collet bore 258. When the collet 122 is coupled with the driving hub 120, distal stops 260 cooperate with flange 244 to prevent the collet from becoming uncoupled yet allow rotation of the hub relative to the collet. Adjacent the proximal end 254, a plurality of proximal stops 262 formed on the collet body 256 protrude inward into the collet bore 258. As seen in FIG. 7B, the proximal stops may be larger than the distal stops, projecting farther into the collet bore. The proximal stops 262 prevent the spring 250 from escaping proximally out of the spring collet 122 and provide a platform against which the spring may be compressed. When coupled in collet 122 with driving hub 120, spring 250 is biased to push the driving hub 120 distally unless otherwise acted upon.

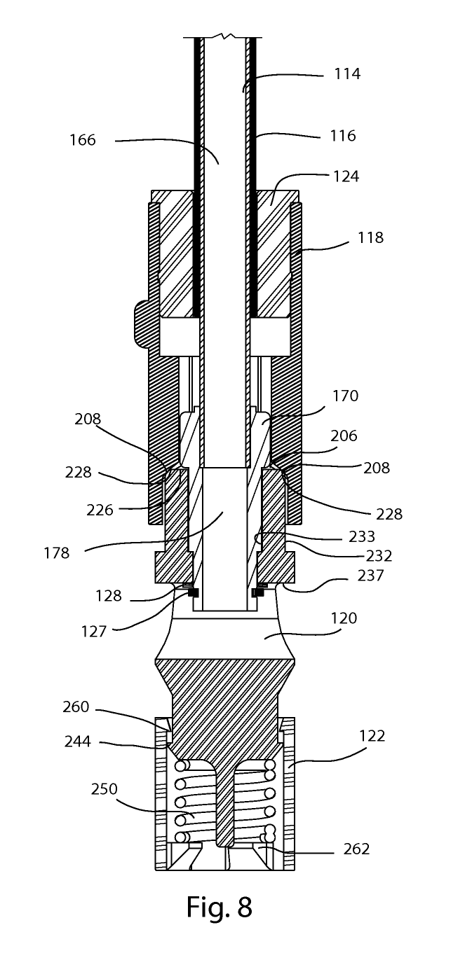

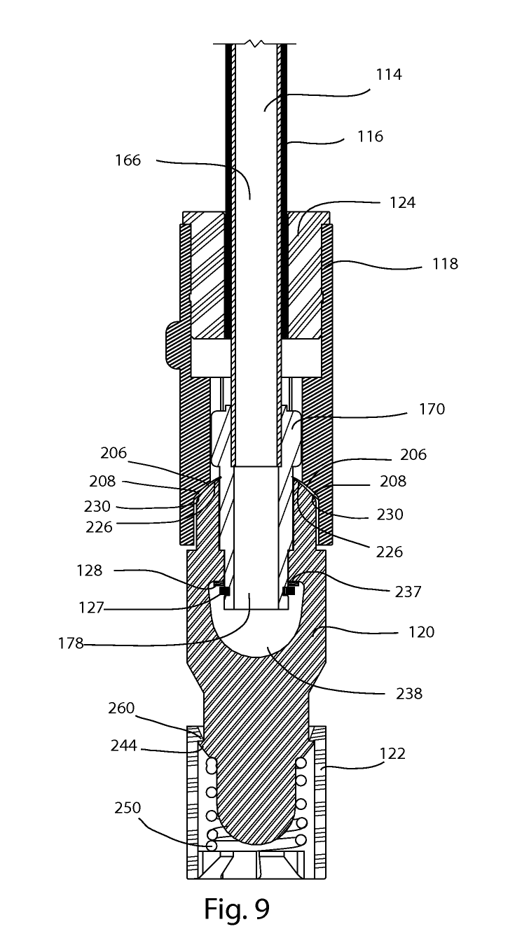

FIGS. 8 and 9 provide cross-sectional views of the handle portion and a segment of the shaft portion of rasp system 100. FIG. 8 shows the rasp system 100 in a retracted configuration, in which the tissue removal member 115 comprising shaft key 170, inner shaft 114 and rasp head 108 is in a first position relative to the outer housing 118. FIG. 9 shows the rasp system 100 in an extended configuration, in which the tissue removal member 115 is in a second position relative to the outer housing 118, the second position distal to the first position. When the rasp system 100 is connected to the powered handpiece and power is supplied, hub 120 is rotated, and the interaction of the cam and cam follower surfaces and the bias of the spring convert the rotary motion of the hub to reciprocal motion of the tissue removal member between the extended and retracted configurations.

As set forth previously, inner shaft 114 is joined with shaft key 170; and shaft key 170 is received within housing 118 such that the wings 184 fit in recesses 204, allowing axial translation of shaft key 170 relative to the outer housing 118 but prohibiting rotation of shaft key 170. A proximal portion of shaft key 170 is received within the driving hub bore 240, which is rotatable relative to the shaft key 170 and the outer housing 118. More specifically, the inner wall 233 slidably rotates about the shaft key 170 while the outer wall 232 slidably rotates relative to the housing 118. The cam surface 206 of the outer housing 118 is positioned immediately adjacent the complementary cam follower surface 226 of the driving hub 120. The cam surface 206 of the outer housing 118 is distal to the proximal end of the tissue removal member 115.

A motion conversion mechanism, which may also be called a motion mechanism, is provided by the outer housing including its cam surface and the hub including its cam follower surface. In extended configuration, hub 120 is positioned such that cam follower surface 226 is flush against cam surface 206, with hollows 230 on follower cam surface 226 complementarily fitting against the lobes 208 of cam surface 206. In the retracted configuration, the driving hub 120 is rotated relative to the outer housing 118 such that the lobes 228 on follower cam surface push against the lobes 208 of cam surface 206, thus forcing driving hub 120 proximally, or downward, relative to the outer housing 118. As hub 120 moves proximally, shaft key 170, inner shaft 114 and rasp head 108 are pulled proximally with the hub, but they do not rotate. Proximal hub face 237 rotatably bears against washer 128, which in turn bears against split ring 127, to pull the tissue removal member 115 proximally. As hub 120 continues to rotate, spring 250 pushes distally to axially translate hub 120 back to the extended position, carrying with it shaft key 170, inner shaft 114 and rasp head 108. In the embodiment depicted in FIGS. 8 and 9, cam surface 206 and cam follower surface 226 each have two lobes and two hollows, so that with one full rotation of hub 120, tissue removal member 115 is twice axially reciprocated. In an alternate embodiment, the cam and cam follower surfaces may have more than two lobes and hollows, so that one rotation of the hub may result in multiple reciprocations. In another alternate embodiment, the cam and cam follower surfaces may each have only one lobe and one hollow, resulting in a single reciprocation per revolution. It is appreciated that while the lobes and hollows depicted herein are rounded, however in other embodiments the lobes and/or hollows may be pointed or sharply angular.

As set forth previously, rasp head 108 comprises uni-directionally oriented teeth, which are oriented proximally toward the suction opening 112. Thus, as tissue removal member 115 reciprocates distally and proximally, the teeth cut into any adjacent tissue as the tissue removal member moves proximally. This proximal cutting action may aid in moving cut tissue debris toward the suction opening. Reciprocation of the flat tissue removal surface 115 against the tissue allows for creation or preparation of a flat surface on the tissue.

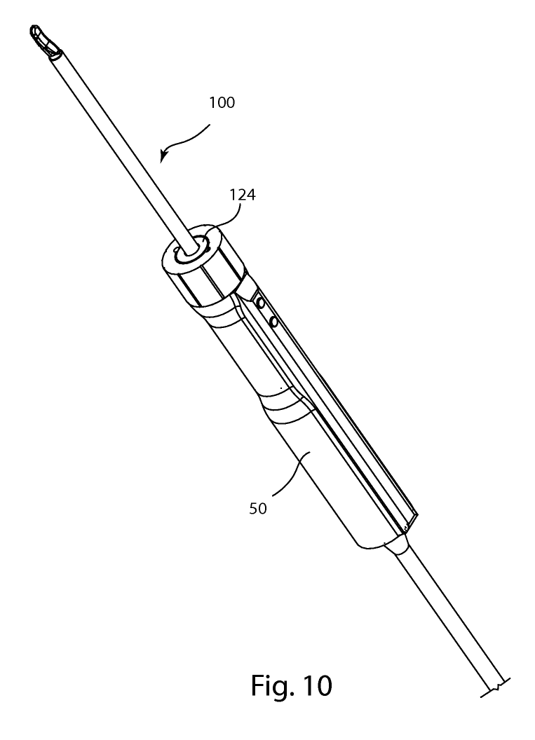

FIG. 10 illustrates rasp system 100 engaged in an exemplary powered rotary handpiece 50. Powered rotary handpiece 50 may be a handpiece known in the art, and provides rotary power and suction to rasp system 100. When the rasp system 100 is engaged in the handpiece, the handle portion 106 is surrounded by the handpiece as in FIG. 10, so that no rotating parts are exposed and so that debris pulled through the suction pathway is captured in the handpiece.

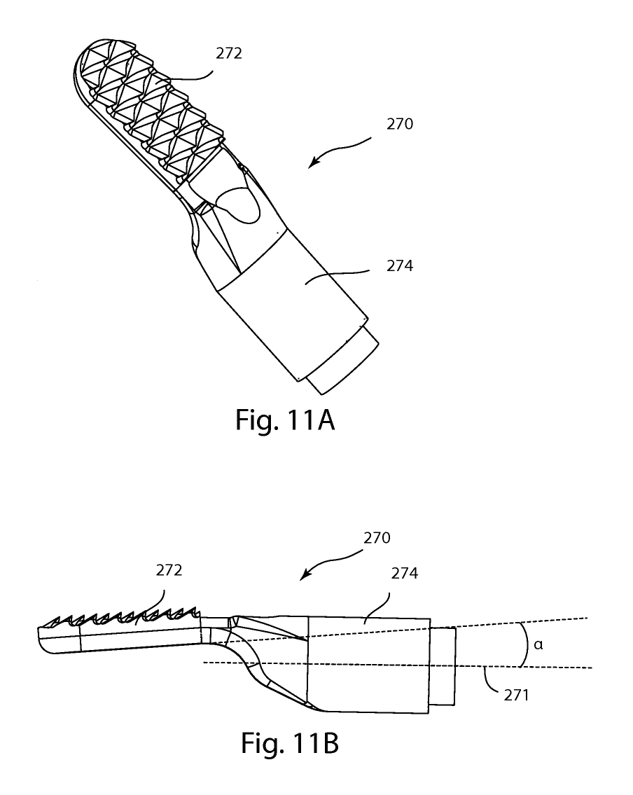

FIGS. 11A through 18B set forth alternate embodiments of the rasp head. It is appreciated that alternate embodiments of the rasp system may include any one of the rasp heads disclosed herein, and may include mixed and matched features of the various rasp heads.

FIGS. 11A and 11B depict a rasp head 270 comprising an angled working portion 272. The working portion 272 is tilted at angle α relative to a longitudinal axis 271 of a head shaft portion 274. Angle α may range from 1 to 10 degrees. More specifically, angle α may range from 3 to 7 degrees. Yet more specifically, angle α may be 5 degrees.

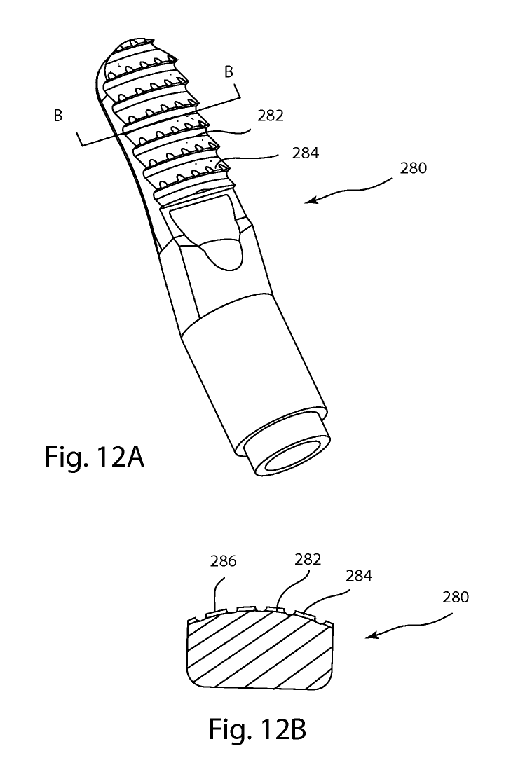

FIGS. 12A and 12B depict a rasp head 280 comprising a convex tissue removal surface 282 from which teeth 284 project. The teeth may comprise straight or curved cutting edges 286; that is the cutting edges 286 may also be convexly curved.

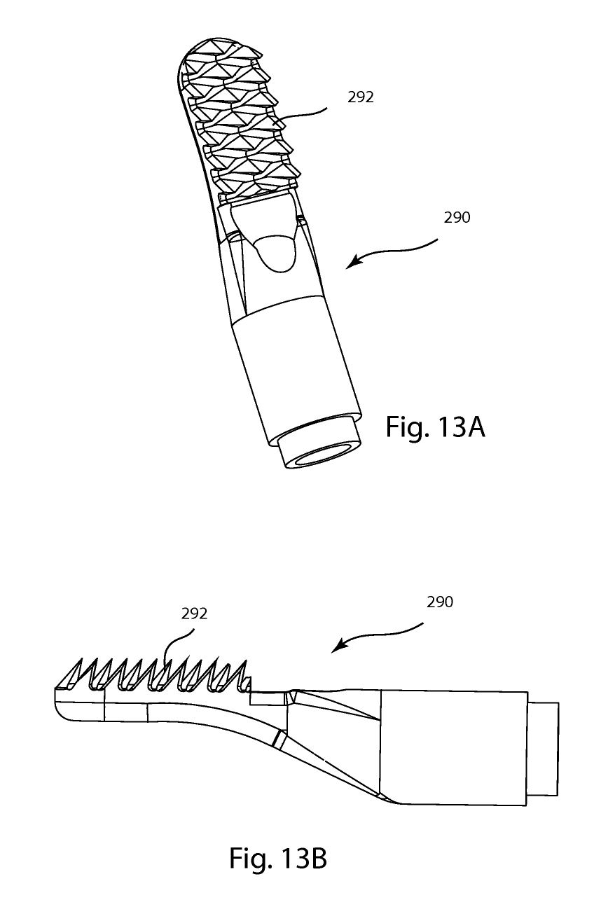

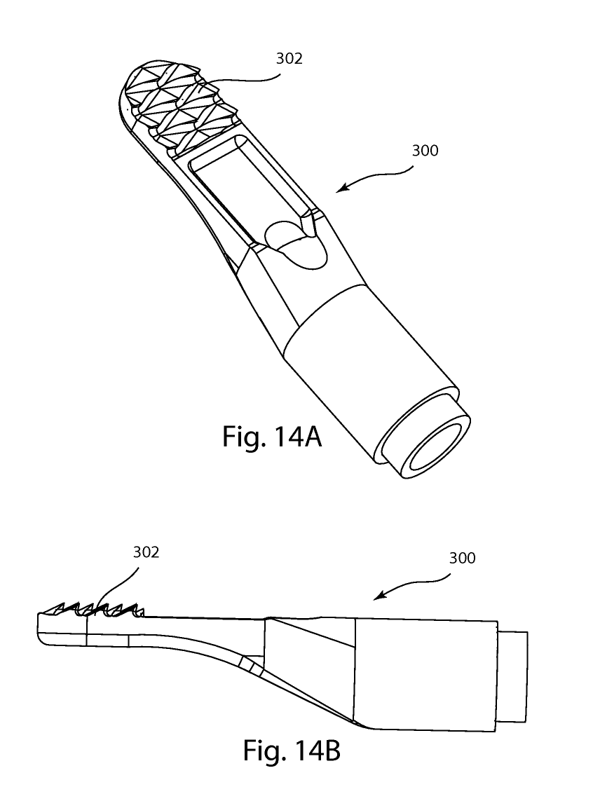

FIGS. 13A and 13B depict a rasp head 290 comprising long teeth 292. The teeth 292 may be longer than teeth in other embodiments and may be advantageous for cutting through relatively softer materials.

FIGS. 14A and 14B depict a rasp head 300 comprising a relatively smaller tissue removal surface 302. This rasp head may be advantageous for accessing smaller and/or more confined areas such as the wrist joint, and for minimizing contact with tissues adjacent the area targeted for tissue removal. It is appreciated that in alternate embodiments, a smaller tissue removal surface may take the form of a longer but narrower tissue removal surface.

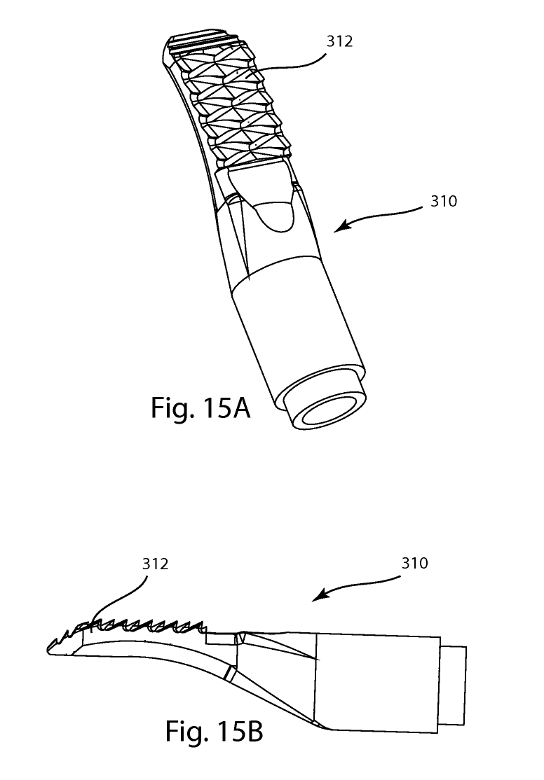

FIGS. 15A and 15B depict a rasp head 310 comprising a curved or crescent-shaped tissue removal surface 312. Tissue removal surface 312 may be convexly curved longitudinally, or both longitudinally and transversely.

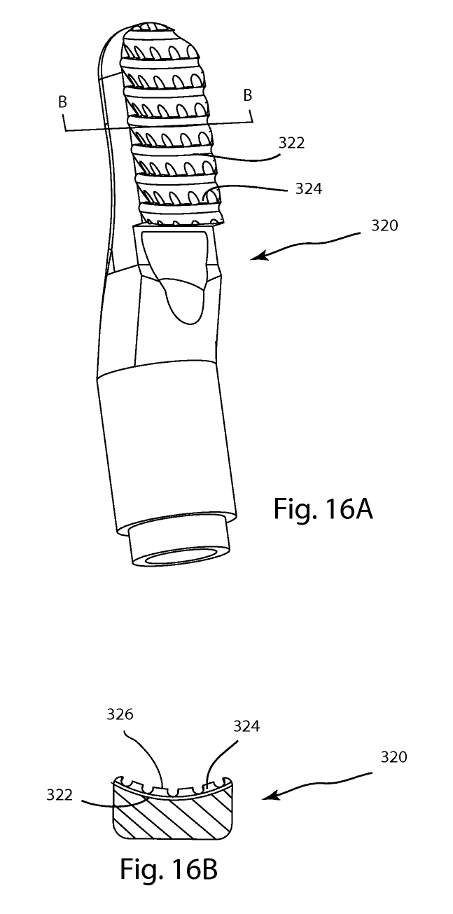

FIGS. 16A and 16B depict a rasp head 320 comprising a concave tissue removal surface 322 from which teeth 324 project. The teeth may comprise straight or curved cutting edges 326; that is the cutting edges 326 may also be concavely curved.

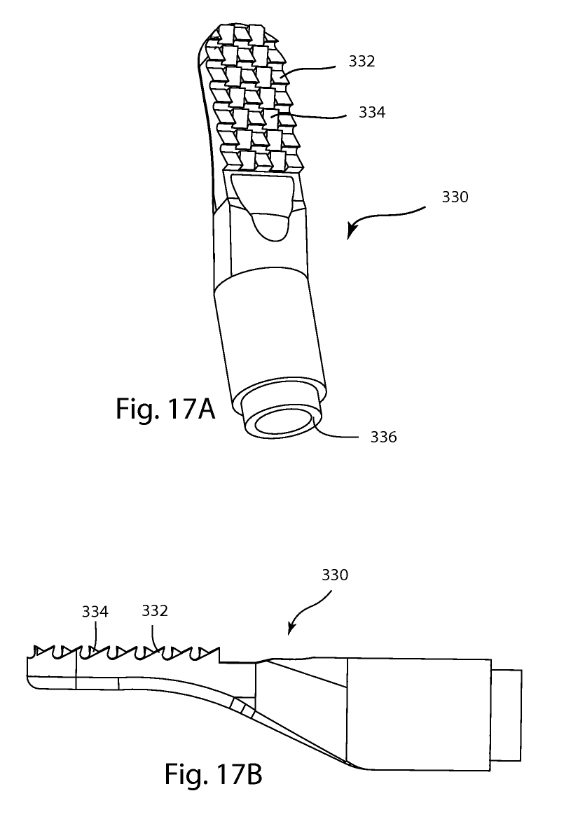

FIGS. 17A and 17B depict a rasp head 340 comprising bi-directional teeth. A plurality of first teeth 332 are oriented proximally, or toward a proximal end 336 of the rasp head, while a plurality of second teeth 334 are oriented distally. When used as part of a reciprocating rasp system such as rasp system 100, tissue cutting may occur in both directions as the rasp head is axially reciprocated.

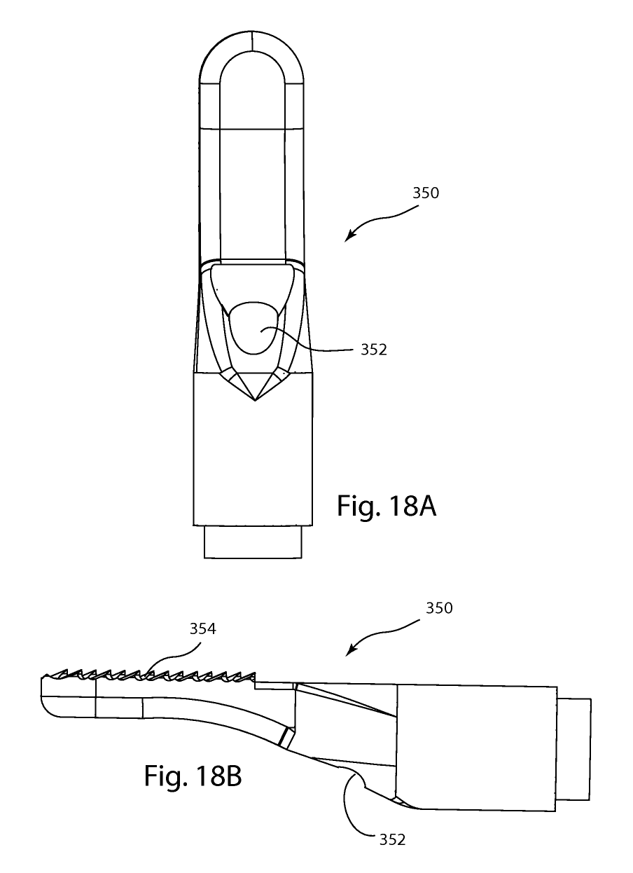

FIGS. 18A and 18B depict a rasp head 350 comprising a suction pathway opening 352 located on the back of the rasp head, on the opposite side as a tissue removal surface 354. It is appreciated that any of the rasp head embodiments disclosed herein may include a similarly located suction pathway opening.

In the embodiments disclosed herein, the rasp head and reciprocating inner shaft may comprise stainless steel, titanium, or other metals or metal alloys. The outer sleeve may comprise metal, plastic, or polymer. The outer housing and rotating hub, and cam and cam follower surfaces, may each comprise polymer, plastic, metal, metal alloy, ceramic, polyether ether ketone (PEEK), thermoplastic polyetherimide (PEI) or a combination thereof. The hub may be coated to improve lubricity or contact strength.

Rasp system 100 may be used in a variety of methods for tissue removal and/or resurfacing. In general, rasp system 100 may be used for abrasionplasty, which encompasses both chondroplasty, or removal of cartilaginous material, and osteoplasty, or removal of bone material. Such tissue removal/resurfacing procedures may be carried out on any bone and/or joint. Similarly, rasp system 100 may be used in treatment of osteochondritis dissecans (OCD) on any affected bone to remove bone fragments. In addition to bone material, rasp system 100 may be used for resurfacing or removal of scar tissue, periosteum, fibrocartilage, functioning cartilage, or nucleus pulposus tissues. Rasp system 100 may also be used in resection and/or resurfacing of bone surfaces in preparation for re-attachment of tendons, preparation for joint fusion, or preparation for implantation of joint replacement device components. The rasp head 108 may be modified to produce alternative embodiments wherein: the size of the rasp head is varied in length, width, and/or thickness; the shape and dimensions of the rasping surface are varied; the number and/or rows of teeth are varied; and/or the orientation of the teeth is varied, among other variations. Rasp 100 and alternative embodiments may be used independently or with common surgical cannulas known in the art. Specific uses for the rasp system 100 and alternative embodiments are set forth herein, however it is appreciated that the rasp may be used in other tissue removal procedures within the scope of the invention.

In the joints of the ankle, rasp system 100 may be used to relieve anterior impingement by removing impinging osteophytes on the talus and/or tibia. Use of rasp system 100 may be advantageous over a bun, as a burr may penetrate too deeply into the bone cortex and cause a fracture in the talar neck. The smaller size and gentler action of rasp system 100 may result in a less aggressive approach than that provided with a bun. Rasp system 100 may also be used in the removal of chondrocytes to address chondromalacia of the talar dome and/or the tibial plafond. Medial and/or lateral guttural impingement of the ankle may be relieved by removal of osteophytes with rasp system 100. Depending on the size, shape and/or accessibility of the tissue to be removed, rasp system 100 comprising rasp head 108 which has a generally flat working surface may be used, or alternative embodiments comprising rasp head 310 with a crescent-shaped working surface or rasp head 280 with a convex working surface may be used.

Rasp system 100 may be used in procedures performed on the knee. Rasp system 100 may be used for symptomatic osteophyte removal, especially along the marginal articular edges of the joint. Rasp system 100 may be used for anterior cruciate ligament (ACL) notch plasty. For this procedure, it may be advantageous to use a system comprising rasp head 310 with a crescent-shaped working surface or rasp head 280 with a convex working surface. Also, a system using rasp head 270 with an angle of 3° to 5° may be ideal for notch plasty access. In addition, rasp system 100 or an alternate embodiment may be used in the knee to perform abrasionplasty to address OCD or chondromalacia.

In the hip, rasp system 100 may be used to address impingement by removal of bony prominences and/or osteophytes. Labral repairs may be performed, such as preparation of the acetabular rim for healing of a labral tear, as a non-limiting example. As in the ankle and knee joints, the rasp may used in the hip for removal of osteophytes and/or chondrocytes to address OCD or chondromalacia. In some procedures in the hip, an alternate embodiment of rasp system 100 comprising a curved shaft portion may be advantageous. In this embodiment the optional outer sleeve may not be required.

In the shoulder, rasp system 100 or alternate embodiments may be used to remove bone and/or cartilage material in at least the following procedures: acromial clavicular joint resection (also known as the Mumford procedure or AC resection); subacromial decompression; glenoid rim abrasionplasty; and osteoplasty in preparation for rotator cuff re-attachment.

In the spine, rasp system 100 may be used in vertebral endplate abrasionplasty, and in preparation for vertebral fusion or artificial disc implantation. Around the facet joints, rasp system 100 may be used for removal of bone spurs, and preparation of articular surfaces for facet joint fusion or replacement. Especially along the curved surfaces around the facet joints, a rasping system comprising the crescent, convex or concave shaped rasp head may be advantageous. Also, the rasp may be used to remove osteophytes or bony prominences in or around the spinal canal.

For procedures in joints of the wrist, a smaller working head surface such as that in rasp head 300 may be advantageous for reaching into confined areas without disturbing adjacent soft tissues. Rasp system 100 may be used for chondroplasty, osteoplasty and other joint preparation procedures in the wrist.

In the elbow, rasp system 100 or alternate embodiments may be used to remove osteophytes on the edges of the trochlea, to prevent impingement on the ulnar nerve. Marginal osteophytes or bony prominences may be removed at the marginal edges of the articulating surfaces of the elbow. For treatment of arthritis, bone spurs may be removed to aid in restoring motion. As with the wrist, use of a system comprising rasp head 300 with a reduced tissue removal surface may be advantageous, as may use of a system comprising a convex or crescent shaped head.

In the skull, rasp system 100 may be employed for sculpting of bony prominences on the cheek areas, forehead, nose, chin and jaw.

Removal of soft tissues adjacent to articular joints is often necessary to gain access to the joint space. For example, in a hip or shoulder arthroscopy procedure, the ligaments forming the joint capsule may need to be resected or penetrated to clear a pathway for a surgical instrument to reach the joint. Disclosed herein are embodiments of a reciprocating rasp system which includes integral RF ablation capability, allowing a practitioner to use a single instrument for RF ablation or coagulation of soft tissues, and removal of hard or bony tissues. The localized RF current flow provided by the instruments disclosed herein may vaporize soft tissues to which it is applied. Use of the combined rasp/RF instrument may provide advantages including: the need for fewer portal incisions, which may reduce patient pain and/or healing time; reduced complexity of the procedure, since fewer individual instruments are required; reduced tissue trauma, as fewer instruments are moved in and out of the affected area, and reduced cost.

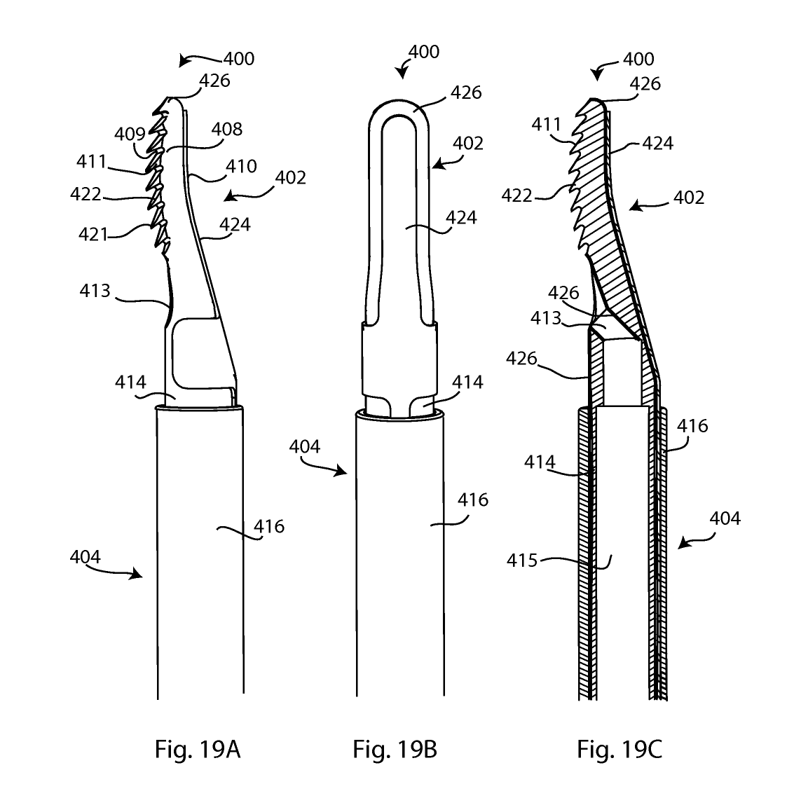

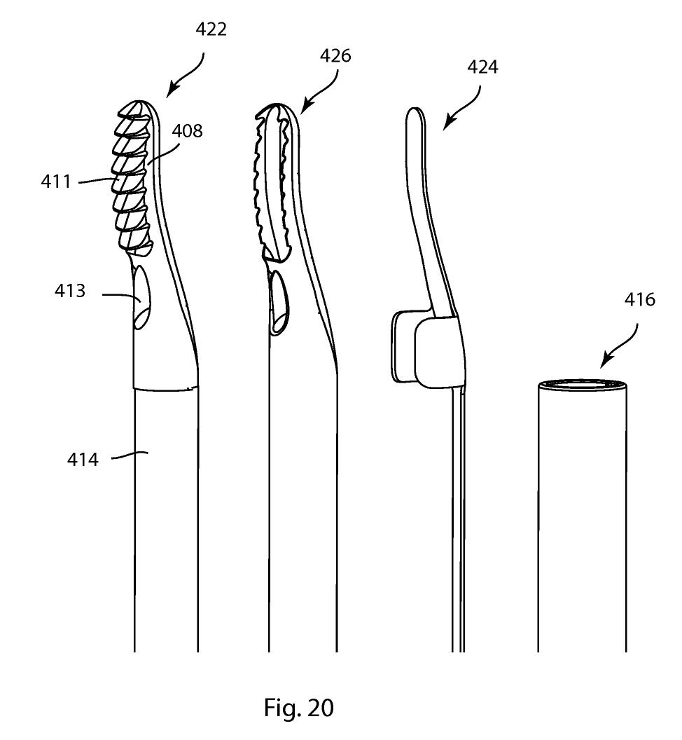

FIGS. 19A-24 illustrate embodiments of reciprocating rasp systems with integrated RF ablation capability. Although not all possible combinations are shown, it is appreciated that an RF/rasp system may include any of the reciprocating rasp variations disclosed herein, with any of the rasp head configurations disclosed herein. Referring to FIGS. 19A-19C and 20, one embodiment of an RF/rasp device 400 includes a head portion 402, shaft portion 404, and handle portion 406 (not shown, but may include the same components as handle portion 106 or other handle portions described herein). The head, shaft and handle portions of system 400 may be the same as other head, shaft and handle portions disclosed herein, with the addition of an RF ablation system 420 integrated into the device. Head portion 402 includes rasp head 408, which has a first side 409 and a second side 410 opposite the first side. A tissue removal surface 411 and suction opening 413 for a suction pathway are located on the rasp head 408. A tissue removal member 412 comprises rasp head 408 and inner shaft 414, and may further include portions of the RF ablation system 420. Shaft portion 404 includes inner shaft 414 and outer sleeve 416, and may further include portions of the RF ablation system 420. A suction pathway 415 comprising distal suction opening 413 and a proximal opening on the hub in the handle portion 406 extends through device 400. Tissue removal surface 411 may comprise a plurality of teeth 421 for cutting and removing hard tissue.

The RF ablation system 420 includes an ablation electrode 422, a return electrode 424, and may include an insulation layer 426 positioned between the ablation and return electrodes. RF system 420 may be described as a bi-polar RF system. In this embodiment of FIGS. 19A-20, the ablation electrode 422 is co-located with the rasp head 408 and inner shaft 414. Insulation layer 426 coats a majority of rasp head 408, except where tissue removal surface 411 protrudes from the insulation, so that when the ablation system is powered or energized, RF energy is transmitted from the tissue removal surface 411, effectively making tissue removal surface 411 the active ablation electrode. The portion of the ablation electrode which protrudes from the insulation may be referred to as the active ablation portion of the ablation electrode. The insulation layer 426 may also coat all or a portion of the length of the inner shaft 414, and may coat a portion of the suction opening 413, as shown. The RF ablation system 420 is connected to a power source and a controller for controlling transmission of RF current through the system. The controller may be a switch, knob, pedal, lever, dial, button or other suitable control member, located on the powered rotary handpiece 50, or on a separate control apparatus. The RF probe may be powered via the controller on to transmit RF current simultaneously with reciprocation of the tissue removal member; alternately, it may be turned on and off independently of tissue removal member reciprocation. The exposed, or uninsulated surface area of the return electrode 424 may be referred to as the active return portion of the return electrode, and is at least three times greater than the exposed surface area, or active ablation portion, of the ablation electrode 422. The ridges of the rasp teeth on the tissue removal surface may enhance arcing of RF current transmitted from the active ablation portion of the ablation probe.

In a method of use, a practitioner may insert head portion 402 into a targeted area, position tissue removal surface 411 adjacent soft tissues to be treated, activate the RF system 420 to ablate or coagulate soft tissue with RF current flow from the ablation electrode 422 to clear a pathway to a joint, turn off the RF system, position the tissue removal surface 411 adjacent hard tissues to be removed, then power the reciprocating motion to use the tissue removal surface 411 to treat adjacent hard tissue. Soft tissues to be removed through ablation or coagulation may comprise muscle, skin, fascia, blood vessels, ligamentous or other relatively soft tissues, while hard tissues may comprise bone, scar tissue, periosteum, fibrocartilage, functioning cartilage, nucleus pulposus tissues, or other relatively hard tissues. The RF current flow may also cauterize blood vessels and/or coagulate blood flow. Alternatively, RF ablation and rasp reciprocation may be powered simultaneously to remove hard and soft tissues at the same time. Suction may be provided as needed, simultaneously with or independently between RF ablation and rasp reciprocation functions. The suction may pick up loose tissue particles or resected pieces of tissue, remove bubbles created by tissue ablation or blood vessel cauterization/coagulation, and/or help maintain visualization of the surgical site. All of these functions may be accomplished without removal of the head portion 402 from the surgical site. Of course, the functions may be accomplished in any desired order and may be repeated as necessary.

The RF current flow may be provided at selected settings, or power levels to produce the desired results, for example, a higher power level may be used to destroy soft tissues while a lower power level is sufficient for cauterization/coagulation of blood vessels. The overall wattage range of the RF system may be 0 to 300 watts. More specifically, a setting or power level for tissue ablation may be three to four times higher than a setting for blood vessel cauterization or blood coagulation. Yet more specifically, a setting for cauterization/coagulation may be 50 watts, and a setting for tissue ablation may be 200 watts.

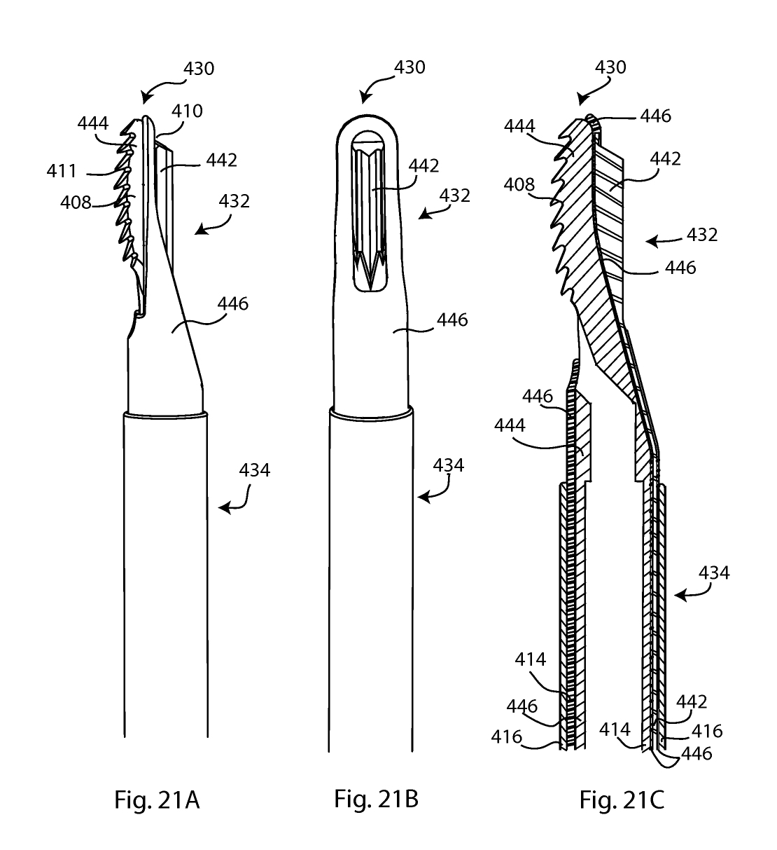

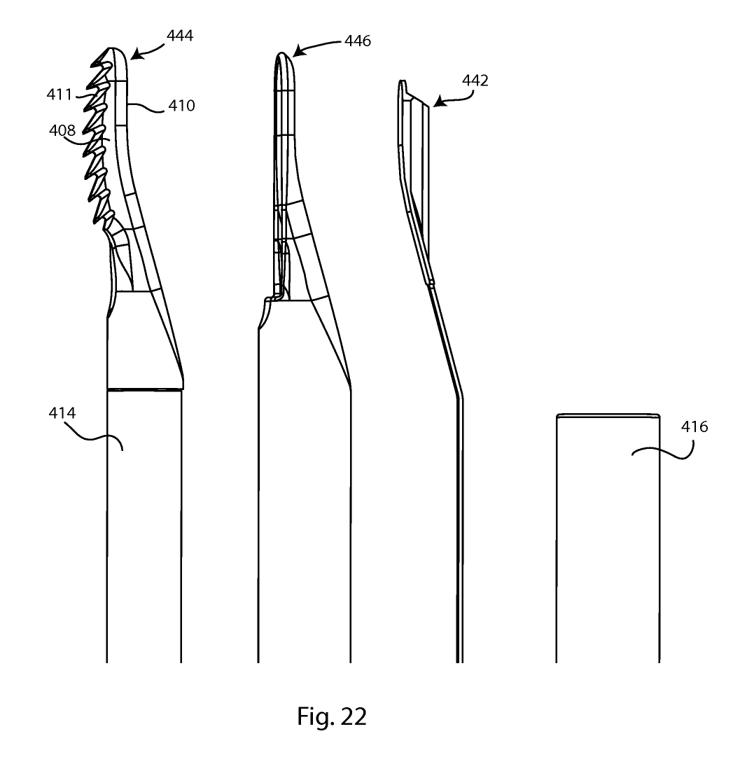

Another embodiment of a reciprocating rasp system with an integral RF ablation system is shown in FIGS. 21A-22. The RF/rasp device 430 includes head portion 432, shaft portion 434, and handle portion 436 (not shown, but may include the same components as handle portion 106 or other handle portions described herein). The device further includes RF ablation system 440, which includes an ablation electrode 442, a return electrode 444, and may include an insulation layer 446 positioned between the ablation and return electrodes. Insulation layer 446 may also be between the ablation electrode 442 and the outer sleeve 416, and between the return electrode 444 and the outer sleeve 416, and may extend the length of the shaft portion 434. In this embodiment of FIGS. 21A-22, the ablation electrode 442 is positioned or carried on a second 410, or back side of rasp head 408. Insulation layer 446 coats a majority of rasp head 408, except where return electrode 444 protrudes from the insulation 446, at tissue removal surface 411, effectively making tissue removal surface 411 the active return electrode. The exposed, or uninsulated surface area of the return electrode 444 is at least three times greater than the exposed surface area of the ablation electrode 442. Ridges or other protrusions formed on the ablation electrode 442 may enhance arcing of electrical energy transmitted from the ablation electrode. The device 430 may further include a suction pathway and suction capabilities as described for other embodiments. Methods of use may be the same as those described for previous embodiments.

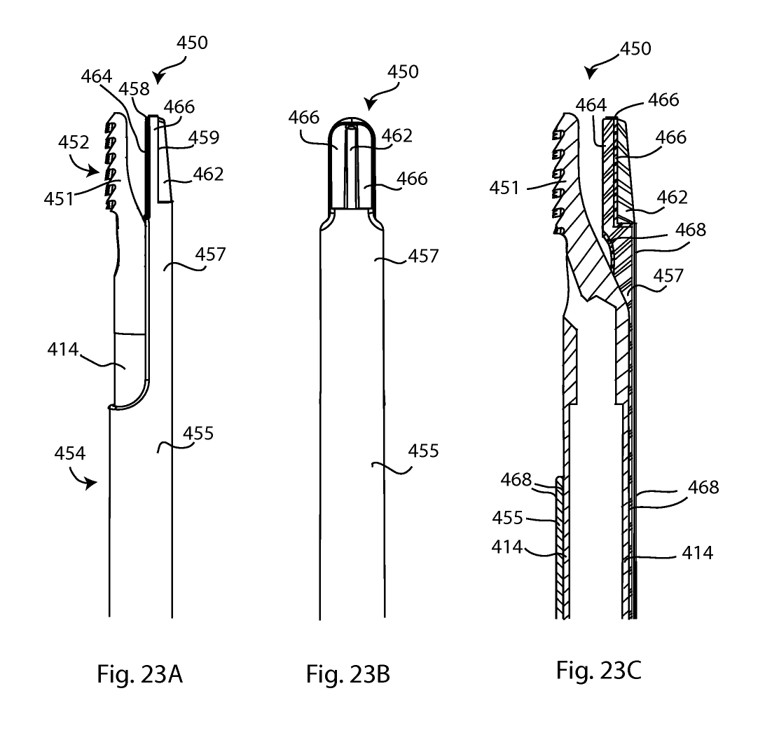

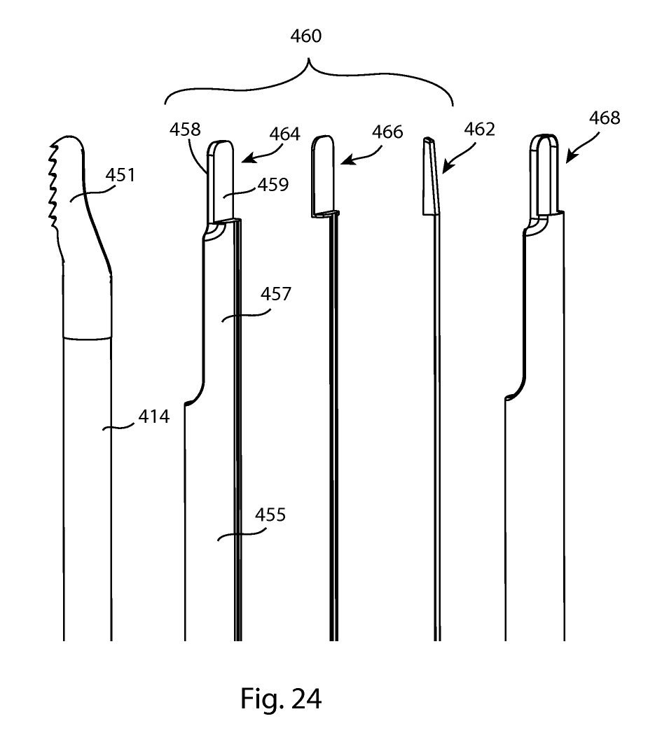

Yet another embodiment of a reciprocating rasp system with an integral RF ablation system is shown in FIGS. 23A-24, in which the ablation and return electrodes are carried on the rasp system stationary outer sleeve. The RF/rasp device 450 includes head portion 452, shaft portion 454, and handle portion 456 (not shown, but may include the same components as handle portion 106 or other handle portions described herein). A reciprocating rasp portion includes rasp head 451 and inner shaft 414. It is appreciated that rasp head 451 may comprise any of the rasp heads disclosed herein and may include features including, but not limited to, teeth or other tissue removal surface, suction opening(s), and a suction pathway. An outer sleeve 455 includes a sleeve extension 457 which projects distally from the tubular portion of the sleeve, and has an inner or first side 458 and an outer or second side 459. The device further includes RF ablation system 460, which includes an ablation electrode 462, a return electrode 464, and an insulation layer 466 positioned between the ablation and return electrodes. In this embodiment of FIGS. 23A-24, the RF system is positioned or carried on the extension 457 of outer sleeve 455. Ablation electrode 462 is carried on the second side 459 of the sleeve extension 457, and may be fin-shaped. Return electrode 464 is integral with sleeve extension 457, and is exposed from the insulation on the first side 458 of the sleeve extension. Insulation layer 466 is sandwiched between the ablation and return electrodes. In this embodiment, sleeve 455 may be coated by a second insulation layer 468 on both the inside and the outside of the sleeve, to isolate it from inner shaft 414. The second insulation layer 468 may also fall outside of the ablation electrode 462, at least along shaft portion 454. Along the shaft portion 454, the ablation electrode 462 is sandwiched between insulation layers 466, 468. The exposed, or uninsulated surface area of the return electrode 464 is at least three times greater than the exposed surface area of the ablation electrode 462. Fins, ridges or other protrusions formed on the ablation electrode 462 may enhance arcing of electrical energy transmitted from the ablation electrode. The device 450 may further include a suction pathway and suction capabilities as described for other embodiments. Methods of use may be the same as those described for previous embodiments.

Suitable materials for the ablation and return electrodes of the RF systems disclosed herein include but are not limited to stainless steel, tungsten, and other conductive materials, metals or metal alloys. Suitable materials for the insulation layers include but are not limited to polytetrafluoroethylene (PTFE), polyolefins, acrylic, polycarbonate, acrylonitrile butadiene styrene (ABS), plastics, and other insulating materials.

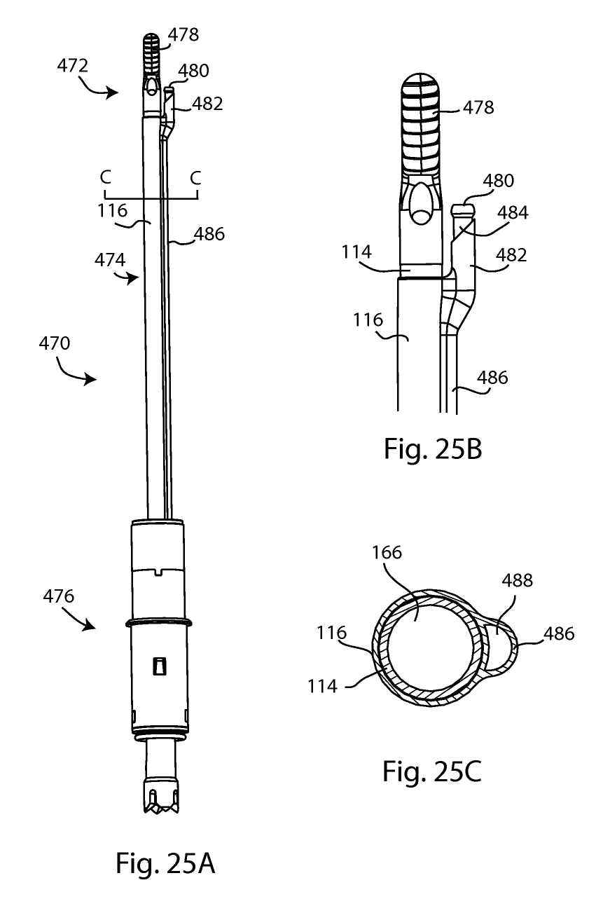

Other embodiments of reciprocating rasp system may include imaging, navigation, and/or infusion capabilities. Referring to FIGS. 25A-25C, rasp system 470 includes imaging and/or navigation capabilities. System 470 comprises head portion 472, shaft portion 474, and handle portion 476. Head portion 472 includes a reciprocating rasp head 478, which may comprise any of the rasp heads disclosed herein, including rasp heads with RF ablation capability. Adjacent head portion 472 is auxiliary device 480. Auxiliary device 480 may be received in a housing 482. Auxiliary device 480 may include an imaging instrument, which may be a camera, ultrasound transmitter, light transmitter, or other imaging transmitter or scanner. In another embodiment, auxiliary device 480 may include a computer-aided navigation reference marker, which may be used in conjunction with a fluoroscopic C-arm and anatomic reference markers to provide intraoperative fluoroscopic images. Auxiliary device 480 may be fixed in housing 482, or may be mobile, able to extend out of housing 482 at any angle. Auxiliary device 480 may be rotatable and sufficiently mobile to capture a 360° view of the environment surrounding the rasp head. For example, auxiliary device 480 may be coupled to a flexible shaft 484, allowing the device 480 to extend and retract in and out of housing 482, and bend around head 478. In the embodiment shown, housing 482 is formed on outer sleeve 116; it is appreciated that the housing may be located at any position relative to head 478, whether laterally adjacent, inferior, or superior to the head. In another embodiment, housing 482 may be integrally formed or co-located with head 478. An auxiliary sleeve portion 486 may be formed on outer sleeve 116 and include an auxiliary bore 488. Wiring and controls for auxiliary device 480 may pass through bore 488.

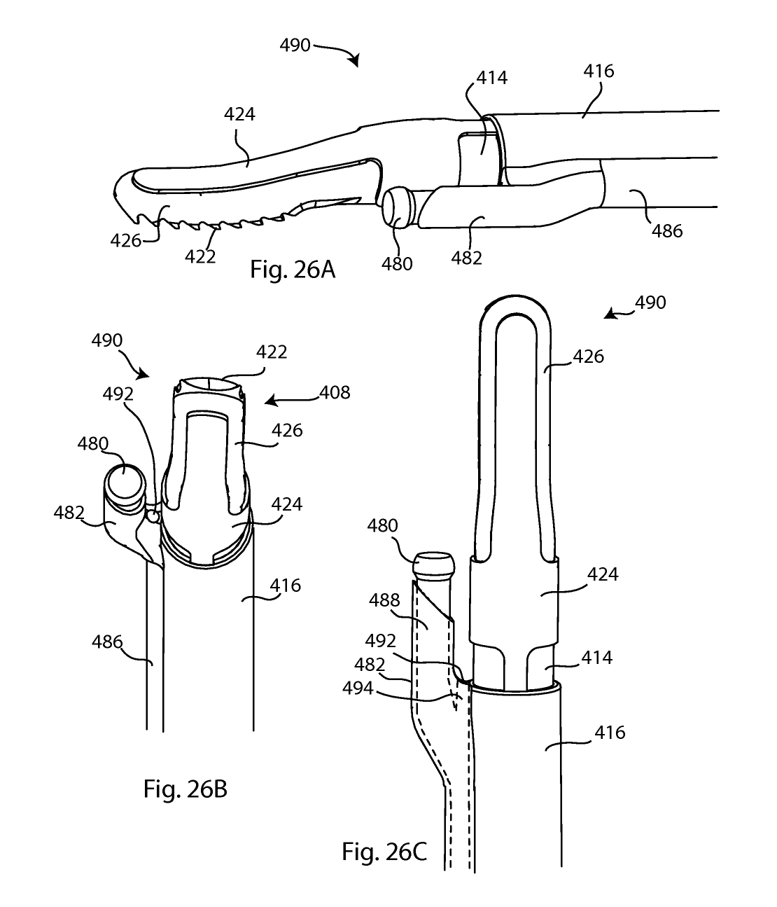

An infusion system may be integrated into any of the rasp systems disclosed herein. FIGS. 26A-26C illustrate one embodiment of such a system. Rasp system 490 includes an RF ablation system 420, auxiliary device 480, and infusion port 492. Infusion port 492 may be positioned adjacent the rasp head 408, providing an opening through which saline or other fluids may be pumped to infuse a targeted site. An infusion bore 494 may open into auxiliary bore 488 as shown or may remain separate. Flexible or rigid tubing may extend through infusion bore 494 to infusion port 492, providing a path for the fluid from a fluid source to the port. By way of non-limiting example, the infusion system may introduce saline, pain relief medication, bone morphogenic protein, bone growth stimulator, anesthetic agents, analgesic agents, anti-inflammatory agents, anti-rejection agents, growth factors, antibiotics, anti-adhesion factors, saline, glycosaminoglycan varieties, collagen varieties, bio-nutrients, gene-delivery vehicles, stem cells, and/or any other therapeutic substance that is desirable to be dispensed to the surgical site. Infusion may be used in conjunction with the suction capabilities of the rasp system, or separately.

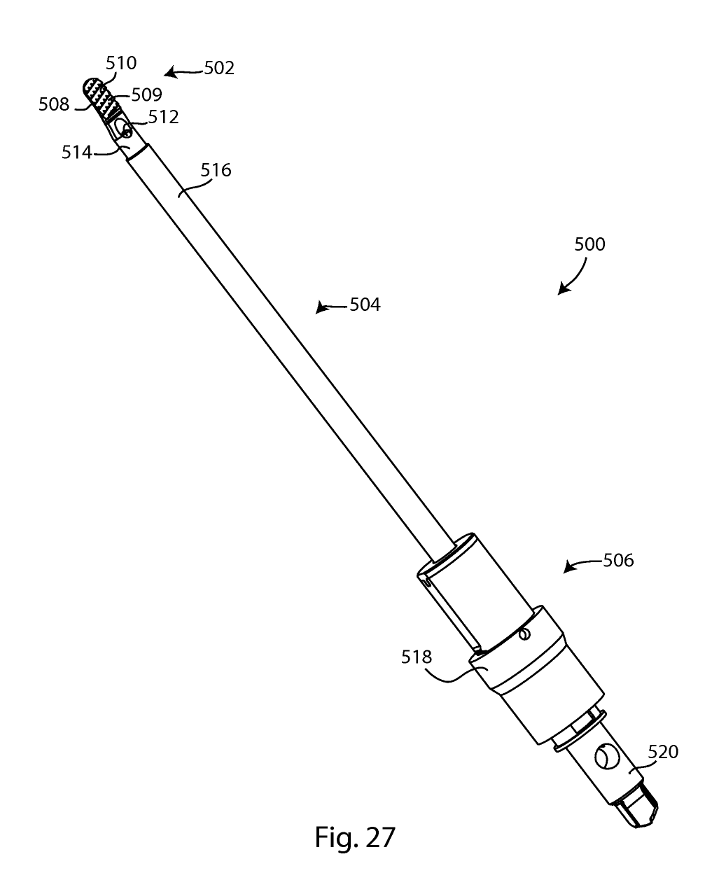

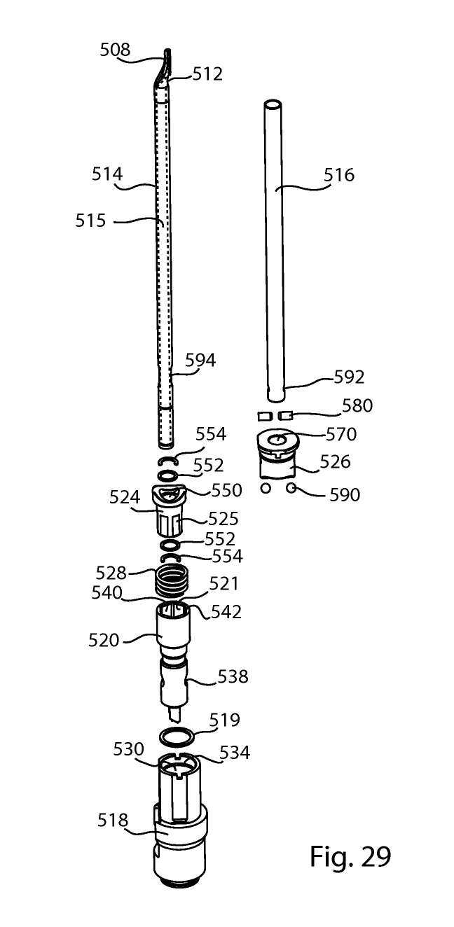

FIGS. 27-33 illustrate an embodiment of a reciprocating rasp system which includes an alternate embodiment of a motion conversion mechanism for converting rotary to reciprocating motion. Referring to FIG. 27, rasp system 500 is shown in an isometric view. Rasp system 500 comprises head portion 502, shaft portion 504, and handle portion 506. Head portion 502 comprises rasp head 508, which includes a tissue removal surface 509 having a plurality of teeth 510 or cutting edges which may cut anatomical tissues when drawn along the tissue surface. A suction opening 512 is located on the head portion 502, and may be disposed between the teeth and the shaft portion. The shaft portion 504 comprises inner shaft 514 which extends proximally from the rasp head 508 and is received in the handle portion 506. Inner shaft 514 is hollow, having a bore 515 (not visible in FIG. 27, seen in FIG. 29) extending from suction opening 512 to a proximal end of the inner shaft, the bore 515 forming a portion of a suction pathway. The inner shaft 514 extends through an optional outer sleeve 516 which is joined to the handle portion 506. Handle portion 506 includes a outer housing 518 which encloses a cam 524 and a fixed cam (within housing 518; not visible in FIG. 27) and partially houses a rotatable hub 520 which is coupled to the cam. When handle portion 506 is engaged in a powered rotary handpiece and power is supplied, hub 520 rotates and consequently cam 524 also rotates, and the cam and fixed cam provide a motion conversion mechanism which converts the rotary motion of the hub to axial reciprocal motion of the inner shaft 514 and attached head 508. Hub 520 may also be referred to as a sluff chamber.

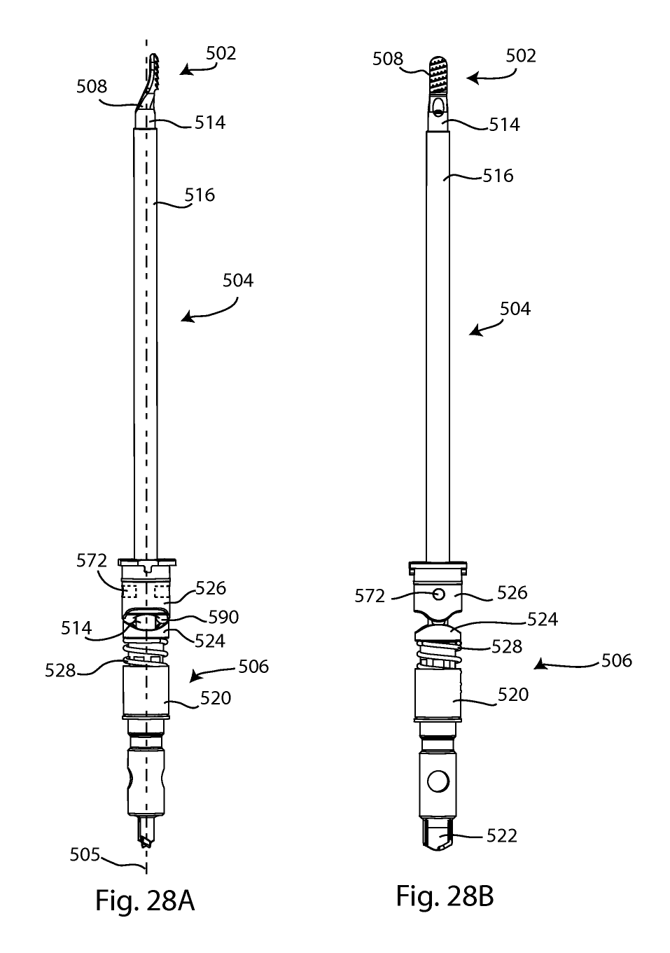

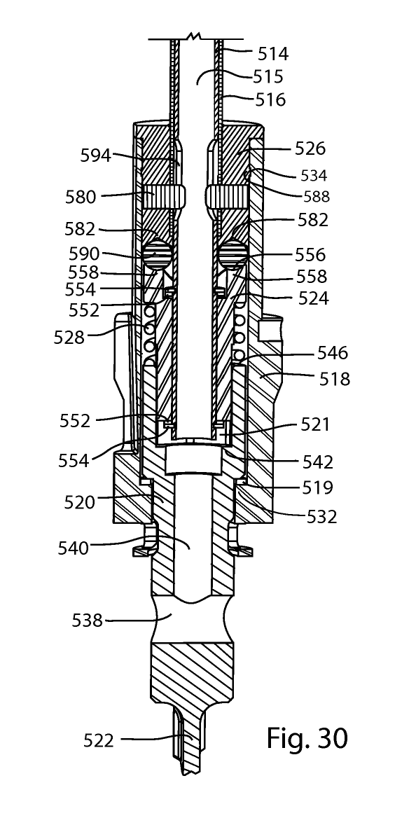

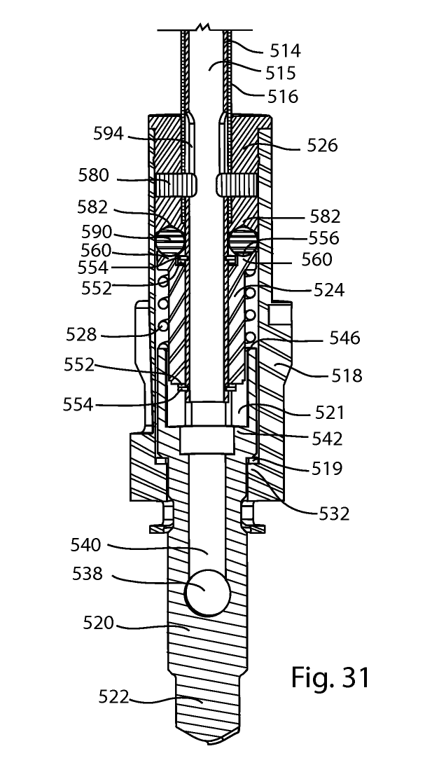

FIGS. 28A and 28B show side and bottom views of rasp system 500, respectively. The outer housing 518 is not shown so that the juxtaposition of the component parts may be seen, relative to longitudinal system axis 505. FIG. 29 is an exploded isometric view of the system. With reference to these drawings, system 500 will be described in a generally proximal to distal sequence. At the proximal end of the assembled system, hub 520 includes a driver connection 522, which may be a tab shaped to be coupled with a driver in a powered handpiece, as described earlier with reference to system 100. When hub 520 is received in housing 518 as in FIG. 27, a coupler washer 519 located between hub 520 and housing 518 promotes free rotation of the hub relative to the housing, thus reducing friction and potentially preventing melting of the two components. Hub 520 further includes a connection feature 521 shaped to receive the cam 524 in a sliding connection, wherein cam 524 is partially captured in hub 520 so that it is rotatably carried with the rotation of hub 520 about longitudinal axis 505, but can also reciprocate along axis 505. The connection feature 521 may be a hex feature, and cam 524 has a corresponding connection feature 525. Inner shaft 514 extends proximally through cam 524, and is slidably engaged with cam 524 so that it does not rotate with cam 524, but is reciprocatively carried with cam 524 between a proximal, or retracted, and a distal, or extended, position. Distal to cam 524, fixed cam 526 may be connected to outer sleeve 516, and may be rigidly connected to housing 518. As cam 524 rotates, cam and cam follower surfaces on cam 524 and fixed cam 526 cooperate to convert the rotary motion of the hub 520 and cam 524 to reciprocating movement of the cam 524 and inner shaft 514. At least one ball bearing 590 may be positioned between the cam and cam follower surfaces, and may reduce friction between the surfaces. A spring 528 is positioned between hub 520 and cam 524, and the spring bias of spring 528 returns the cam 524, inner shaft 514 and rasp head 508 to the distal position. It is appreciated that in other embodiments, the relative sequence of the system components may vary to accomplish the same objectives. For example, in another embodiment the relative positions of the cam 524 and fixed cam 526 may be reversed, or the location of the spring 528 may differ. Referring to FIGS. 30 and 31, longitudinal cross-sectional views show the handle portion 506 of system 500 in the retracted and extended positions, respectively. With reference to FIGS. 29-31, system 500 is described in more detail. Housing 518 has a generally elongated tubular shape, and may include external engagement features such as slots, grooves, tabs or faces shaped for engagement with a specific powered handpiece. Housing 518 may be referred to as an adapter body. A housing bore 530 formed in housing 518 is smooth sided to allow free rotation of hub 520 and cam 524 within the bore, and free reciprocation of cam 524. A housing shoulder 532, formed as a step in bore 530, provides a seat for coupler washer 519, and retains hub 520 partially within the housing. Near the distal end of housing 518, a housing groove 534 may be formed in bore 530 for retention of fixed cam 526.

Hub 520 has a generally elongated, and partially tubular form. A transverse bore 538 is formed toward a proximal end of the hub, and a longitudinal bore 540 is formed from a distal end of the hub, extending longitudinally into a portion of the hub and opening into the transverse bore 538. The longitudinal 540 and transverse 538 bores form a segment of the suction pathway. The inside diameter of the longitudinal bore 540 is stepped, and in other embodiments may be tapered. One step forms a first hub shoulder 542, which may provide a proximal stop for reciprocation of cam 524. Another step forms a second hub shoulder 544, which may provide a proximal stop for reciprocation of inner shaft 514. A distal portion of longitudinal bore 540 is connection feature 521, which may be a hex as previously set forth. A distal end 546 of the hub 520 provides a platform or seat for spring 528.

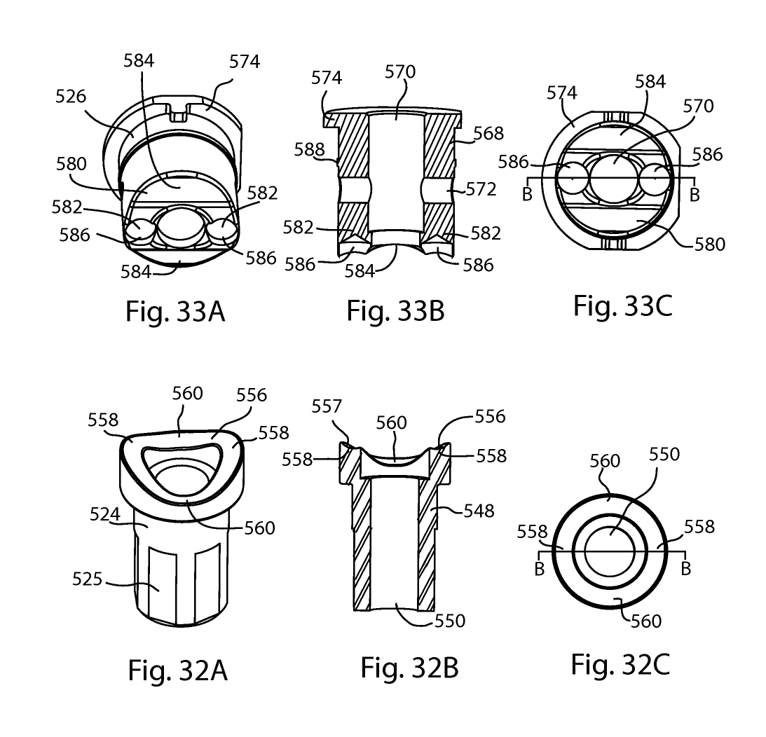

Cam 524 has a generally elongated tubular body 548, and is sized so that a proximal portion is received in longitudinal bore 540 of hub 520. When assembled, the cam 524 may be entirely enclosed in housing 518. A cam bore 550 extends longitudinally through the length of the cam body 548, and is sized to receive inner shaft 514. When inner shaft 514 is positioned in cam bore 550, a washer 552 and snap ring 554 are placed around inner shaft 514 at each end of cam 524, the snap rings 554 fitting into grooves formed in the inner shaft 514 to retain cam 524 in a fixed longitudinal position relative to inner shaft 514, while simultaneously allowing free rotation of cam 524 relative to shaft 514. Further detail of cam 524 is seen in FIGS. 32A-C. A portion of the outer surface of cam 524 forms connection feature 525, which is shaped to complementarily engage connection feature 521 on hub 520. Although hex shaped connection features are shown in the figures, it is appreciated that in other embodiments the connection features could comprise other complementary shapes. Toward the distal end of cam 524, cam surface 556 is formed on cam 524. Cam surface 556 is generally annular or circular and undulating, forming two protruding lobes, or high points 558 alternating with two low points 560. The high and low points are evenly distributed; the high points at 180° from each other and the low points at 180° from each other, and the low points 90° from each high point. It is appreciated that in other embodiments of the invention, the cam surface 556 could have one high and one low point; or multiple high and low points. The cam surface 556 may be recessed, forming a grooved track 557, which may be hemispherically grooved. The annular cam surface 556 may also be radially sloped such than the inner diameter of the annulus is lower than the outer diameter at any radial cross-section of the cam surface, as seen in FIG. 32B.

FIGS. 33A-C show further detail of fixed cam 526. Fixed cam 526 has a generally tubular body 568, and includes a fixed cam bore 570 which extends longitudinally through the length of the fixed cam. The bore 570 is sized to receive outer sleeve 516 in a press fit engagement. At least one slot 572 extends through body 568, and is shaped to receive a pin or screw for fixing the position of outer sleeve 516 relative to the fixed cam 526. Toward the distal end of the fixed cam 526, a rim 574 projects from the cam body 568. At the proximal end of the fixed cam 526 is formed a fixed cam surface 580. Fixed cam surface 580 is generally circular and undulating, forming two protruding lobes, or high points 582 alternating with two low points 584. The high and low points are evenly distributed; the high points at 180° from each other and the low points at 180° from each other, and the low points 90° from each high point. It is appreciated that in other embodiments of the invention, the fixed cam surface 580 could have one high and one low point; or multiple high and low points, and that the high and/or low points may be unevenly distributed. At each of the two high points 582, a recessed dimple 586 is formed. The dimples 586 are shaped to partially receive bearings 590.

With reference to FIGS. 27-31, when assembled in system 500, fixed cam 526 is at least partially enclosed by housing 518, and is displaced from hub 520. A rib 588 may be formed on the fixed cam body 568, shaped to fit into housing groove 534. Pins 580 extend through slots 572, through openings 592 in outer sleeve 516, and into elongated slots 594 in inner shaft 514. The pins and slots form a keyway system which fixes the positions of fixed cam 526 and outer sleeve 516 relative to one another, and forms a sliding connection to inner shaft 514. The elongated slots 594 allow inner shaft 514 to reciprocate relative to outer sleeve 516, constrained by pins 580.

In one method of use, handle portion 506 is fitted into a powered handpiece, with driver connection 522 engaging with a rotating driver in the handpiece. When powered on, hub 520 rotates, and cam 524 rotates with hub 520. As cam 524 rotates, cam surface 556 rotates, bearing against bearings 590 retained in dimples 586 of fixed cam 526. During rotation, when the cam high points 558 are aligned with fixed cam high points 582, inner shaft 514 and rasp head 508 are pulled proximally to a retracted position by cam 524, as seen in FIG. 30. Spring 528 is compressed between cam 524 and hub distal end 546. As rotation continues, cam low points 560 become aligned with fixed cam high points 582, and inner shaft 514 and rasp head 508 are pushed distally to an extended position by the spring bias of spring 528, as seen in FIG. 31. In this embodiment, two such retraction-extension cycles are completed with each full rotation of the hub 520. During the cycles, bearings 590 are rotated within dimples 586 as cam surface 556 spins against the bearings. The hemispherical shapes of the cam surface 556 and fixed cam surface 580, and the complementary spherical shape of bearings 590 may provide continual surface contact between the bearings and the opposing cam surfaces.

One way to view the teachings set forth above is to characterize certain structures as tissue removal means. In the various embodiments set forth above the tissue removal means can be said to be element 140 as shown in FIGS. 3A and 3B, or element 272 in FIGS. 11A and 11B, or element 292 in FIGS. 13A and 13B, or element 302 in FIGS. 14A and 14B, or element 312 in FIGS. 15A and 15B, or element 322 in FIGS. 16A and 16B, or element 332 in FIGS. 17A and 17B, or element 354 in FIGS. 18A and 18B, or element 411 in FIGS. 19A-22, or element 509 in FIG. 27, and the tissue removal surfaces seen in FIGS. 23A, 23C, 24, 25A, 25B, 26A. Other tissue removal means are contemplated within the scope of the invention, including but not limited to tissue removal surfaces comprising teeth, ridges, or sharpened or roughened surfaces.

Certain aspects of the teaching set forth above can be characterized as motion conversion means for converting rotary motion of a tissue removal member to reciprocal motion. Structure for the motion conversion means is found in at least FIGS. 8 and 9 in elements 118 and 120, and in FIGS. 30 and 31 in elements 526 and 524. Other motion conversion means are contemplated within the scope of the invention, including but not limited to a rotating hub or sluff chamber and a fixed housing, a cam and a cam follower, and complementary cam and cam follower surfaces.

Certain aspects of the teaching set forth above can be characterized as soft tissue ablation or coagulation means for destroying or coagulating soft tissue. Structure for the soft tissue ablation or coagulation means is found in at least FIGS. 19A-24, and FIGS. 27A-C in RF ablation electrodes 422, 442 and 462. Other soft tissue ablation means are contemplated within the scope of the invention, including but not limited to other RF current emitting elements, electrical energy emitting elements, heat emitting elements, microwave emitting elements, and other energy emitting elements capable of selectively destroying or coagulating soft tissue.

Certain aspects of the teaching set forth above can be characterized as means for suction. Structure for suction means is found in at least FIGS. 3-9 in elements 112, 148, 166, 178, and 238. Another structure for suction means is also found in at least FIGS. 27, 30 and 31 in elements 512, 515, 540 and 538. Other suction means are contemplated within the scope of the invention, for example any suction opening found on any of the rasp heads disclosed herein may form a suction pathway in combination with suction pathway elements of the any inner sleeves and hubs disclosed herein.

It should be understood that the present system, kits, apparatuses, and methods are not intended to be limited to the particular forms disclosed. Rather, they are to cover all modifications, equivalents, and alternatives falling within the scope of the claims.

The claims are not to be interpreted as including means-plus- or step-plus-function limitations, unless such a limitation is explicitly recited in a given claim using the phrase(s) “means for” or “step for,” respectively.

The term “coupled” is defined as connected, although not necessarily directly, and not necessarily mechanically.

The use of the word “a” or “an” when used in conjunction with the term “comprising” in the claims and/or the specification may mean “one,” but it is also consistent with the meaning of “one or more” or “at least one.” The term “about” means, in general, the stated value plus or minus 5%. The use of the term “or” in the claims is used to mean “and/or” unless explicitly indicated to refer to alternatives only or the alternative are mutually exclusive, although the disclosure supports a definition that refers to only alternatives and “and/or.”

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a method or device that “comprises,” “has,” “includes” or “contains” one or more steps or elements, possesses those one or more steps or elements, but is not limited to possessing only those one or more elements. Likewise, a step of a method or an element of a device that “comprises,” “has,” “includes” or “contains” one or more features, possesses those one or more features, but is not limited to possessing only those one or more features. Furthermore, a device or structure that is configured in a certain way is configured in at least that way, but may also be configured in ways that are not listed.

The present invention may be embodied in other specific forms without departing from its spirit or essential characteristics. It is appreciated that various features of the above-described examples can be mixed and matched to form a variety of other alternatives. For example, any rasping head may be combined with any handle portion or driving hub configuration. Similarly, suction, RF ablation, infusion, and/or imaging capability may be included with any rasping system disclosed herein. As such, the described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

1. A tissue removal device for being driven by a powered rotary handpiece, comprising:
a rotary hub;
a tissue removal member comprising a head portion having a first side and a second side opposite the first side, a tissue removal surface located on the first side, the tissue removal member coupled in sliding contact with the rotary hub;
a motion conversion mechanism, wherein when the rotary hub is rotated about an axis, the motion conversion mechanism urges motion of the tissue removal member along the axis, wherein the motion consists of reciprocating translation between a first retracted position and a second extended position; and
a radiofrequency ablation system carried on the tissue removal device for ablation or coagulation of soft tissue, the radiofrequency ablation system comprising an ablating electrode and a return electrode.

2. The tissue removal device of claim 1, wherein the radiofrequency ablation system further comprises an insulation layer positioned between the ablating electrode and the return electrode.

3. The tissue removal device of claim 1, wherein the radiofrequency ablation system is carried on the tissue removal member.

4. The tissue removal device of claim 3, wherein the head portion first side carries the ablating electrode.

5. The tissue removal device of claim 3, wherein the head portion second side carries the ablating electrode.

6. The tissue removal device of claim 3, further comprising a suction pathway extending from a distal opening on the head portion to a proximal opening, the distal opening located on the same head side as the ablating electrode.

7. The tissue removal device of claim 3, further comprising a suction pathway extending from a distal opening on the head portion to a proximal opening, the distal opening located on the opposite head side from the ablating electrode.

8. The tissue removal device of claim 1, further comprising a sleeve member having a distal end and a proximal end, the tissue removal member extending through the sleeve member, wherein the radiofrequency ablation system is carried on the sleeve member.

9. The tissue removal device of claim 8, further comprising a suction pathway extending from a distal opening on the head portion to a proximal opening.

10. A tissue removal device for being driven by a powered rotary handpiece, comprising:
a rotary hub;
a tissue removal member comprising tissue removal means, the tissue removal member coupled in sliding contact with the rotary hub;
means for motion conversion, wherein when the rotary hub is rotated about an axis, the means for motion conversion urges motion of the tissue removal member along the axis, wherein the motion consists of reciprocating translation between a first retracted position and a second extended position; and
means for soft tissue ablation or coagulation carried on the tissue removal device.

11. The tissue removal device of claim 10, further comprising means for suction carried on the tissue removal device.

12. The tissue removal device of claim 10, wherein the means for soft tissue ablation or coagulation is carried on the tissue removal member.

13. The tissue removal device of claim 10, further comprising a sleeve member having a distal end and a proximal end, the tissue removal member extending through the sleeve member, wherein the means for soft tissue ablation or coagulation is carried on the sleeve member.

14. A method for removal of hard and soft body tissues at a surgical site using a single instrument, the method comprising:
providing a tissue removal device for being driven by a powered rotary handpiece, the tissue removal device comprising: a rotary hub, a tissue removal member comprising a tissue removal surface, the tissue removal member coupled in sliding contact with the rotary hub, a motion conversion mechanism, wherein when the rotary hub is rotated about an axis, the motion conversion mechanism urges motion of the tissue removal member along the axis, wherein the motion consists of reciprocating translation between a first retracted position and a second extended position, and a radiofrequency ablation system comprising an ablating electrode and a return electrode;
positioning the tissue removal surface adjacent hard tissue to be removed;
powering the tissue removal device to provide the reciprocating translation of the tissue removal surface to remove a selected amount of hard tissue;
positioning the ablating electrode adjacent soft tissue to be ablated or coagulated; and
powering the radiofrequency ablation system to ablate or coagulate a selected amount of soft tissue by radiofrequency ablation.

15. The method for removal of hard and soft tissues of claim 14, wherein the tissue removal device further comprises a suction pathway, the method further comprising:
applying suction to suction the removed tissue through the suction pathway away from the surgical site.

16. The method for removal of hard and soft tissues of claim 14, further comprising:
powering the radiofrequency ablation system to ablate or coagulate soft tissue while simultaneously powering the tissue removal device to provide reciprocating translation of the tissue removal surface.

17. The method for removal of hard and soft tissues of claim 14, wherein the ablating electrode is carried on the tissue removal member, the tissue removal member comprising a head portion having first side and a second side opposite the first side, the tissue removal surface located on the first side.

18. The method for removal of hard and soft tissues of claim 17, wherein the ablating electrode is carried on the tissue removal surface, the method further comprising:
positioning the tissue removal surface adjacent soft tissue to be ablated or coagulated.

19. The method for removal of hard and soft tissues of claim 17, wherein the ablating electrode is carried on the head portion second side, the method further comprising:
positioning the head portion second side adjacent soft tissue to be ablated or coagulated.

20. The method for removal of hard and soft tissues of claim 14, wherein the tissue removal device further comprises a sleeve member having a distal end and a proximal end, the tissue removal member extending through the sleeve member, wherein the radiofrequency ablation system is carried on the sleeve member, the method further comprising:
positioning the sleeve member adjacent soft tissue to be ablated or coagulated.
